SEQUENCE LISTING (44 sequences)

SEQ ID NO: 1 - protein, 489 aa, synthetic construct
  REGION 1..489: note = Amino acid sequence of anti-hCD19 CAR variant 1
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 467 aa, synthetic construct
  REGION 1..467: note = Amino acid sequence of anti-hCD19 CAR variant 2
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGGSGGLITRLWCKLDSPASTKWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 657 aa, synthetic construct
  REGION 1..657: note = Amino acid sequence of anti-hCD19 CAR variant 3
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQV

SEQ ID NO: 4 - protein, 820 aa, synthetic construct
  REGION 1..820: note = Amino acid sequence of anti-hCD19 CAR variant 4
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 5 - protein, 465 aa, synthetic construct
  REGION 1..465: note = Amino acid sequence of anti-hCD19 CAR variant 5
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 467 aa, synthetic construct
  REGION 1..467: note = Amino acid sequence of anti-hCD19 CAR variant 6
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 472 aa, synthetic construct
  REGION 1..472: note = Amino acid sequence of anti-BCMA CAR variant 1
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 467 aa, synthetic construct
  REGION 1..467: note = Amino acid sequence of anti-BCMA CAR variant 2
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 1470 bp, synthetic construct
  source 1..1470: mol_type = other DNA
  misc_feature 1..1470: note = Nucleotide sequence of anti-hCD19 CAR variant 1
atgctgctgttggtgacttctctcctcctgtgtgagctgccccaccctgcctttctgctcatcccagacatccagatgacccagaccaccagcagcctgtctgcctccctgggggacagagtgaccatctcctgccgggccagccaggacatttccaagtacctcaactggtaccagcagaagccagatggaactgtcaaacttctgatctaccacacctcccggctccacagcggggtgcccagccgcttcagcggctctggcagtgggacagactattccctcaccatcagcaacttggagcaggaagatattgccacctacttctgccagcaaggcaacaccctgccttacacctttggaggaggcaccaagctggaaattacaggctccaccagtggatctggaaaacctggctcaggagaaggaagcaccaaaggagaggtgaaactgcaagaaagtggccctggacttgtggctccatcccagagcctcagcgtcacctgcaccgtgtctggggtcagccttcctgattatggagtgtcctggatccggcagcctccaagaaaagggctggagtggctgggtgtgatctggggaagtgagaccacttactacaattcagccctcaagtccaggctgaccattatcaaggacaacagcaaatctcaggtgttcctgaagatgaattctctgcagacagatgacacagccatctattactgcgccaagcactactattatggcggcagctatgccatggactactggggccagggcacatctgtgacagtgagctctgctgccgccattgaagtgatgtaccccccaccctacctggacaatgagaagagcaatggaacaatcattcatgtgaagggcaagcatctgtgccccagtcccctcttcccaggcccctccaaaccattttgggtcctggtggtggtcgggggcgtgctggcctgctacagcttgctggtcactgtggccttcatcatcttctgggtgcgcagcaagaggagccggctgctgcattctgattacatgaacatgacaccccggcggcctgggcccaccaggaaacattatcagccctatgctcctcctcgggactttgctgcttatagaagcagggtcaagttctccagatctgcagatgccccagcctaccagcaggggcagaaccagctgtataatgaactgaacctgggccgccgggaggagtatgacgtgttggacaagagaagaggaagagatcctgagatgggcgggaagcccaggaggaagaacccccaggagggcctctacaacgagctccagaaagataaaatggccgaggcctattctgagatcggcatgaagggggagcggcgccgcgggaaaggccatgatggcctgtaccaaggactgagcactgccacaaaagacacctatgatgctctgcacatgcaggccctgccaccaaggtga

SEQ ID NO: 10 - DNA, 1407 bp, synthetic construct
  source 1..1407: mol_type = other DNA
  misc_feature 1..1407: note = Nucleotide sequence of anti-hCD19 CAR variant 2
atggacatccagatgacccagaccaccagctccctgtctgcctctctgggggacagagtcaccatctcctgccgggccagccaggacatttccaagtacctcaactggtaccagcagaagccagatggcactgtcaaactcctgatctaccacacctccaggctgcattctggagtgcccagccgcttcagtgggagcggctctggcacagactattccctcaccatcagcaacttggagcaggaagatattgccacctacttctgccagcaaggcaacaccctgccctacacctttggaggagggaccaaactggagatcacaggcagcacctcaggctcaggaaaacctggaagtggagaaggaagcacaaaaggagaggtgaaacttcaagaaagtggccctgggctggtggctccttcccagagcctcagcgtgacctgcaccgtgtctgggggcagtggtggcctcatcacccgcctgtggtgcaagctggattctcctgcctccaccaagtggctgggagtcatctggggatctgagaccacttactacaattcagccctcaagtccagactgaccattattaaagacaacagcaaatctcaggtgttcctgaagatgaattctctccagacagatgacacagccatctattactgcgccaagcactactattatggaggctcctatgccatggattactggggccagggcacctctgtgacagtgtcctcagcagctgccattgaagtgatgtacccacctccttacctggacaatgagaagagcaatggaacaatcattcatgtgaagggcaagcatctgtgccccagtcctctcttcccagggccctccaaaccattttgggtcctggttgtggtgggcggcgtgctggcctgctacagcctgctggtcactgtggccttcatcatcttctgggtgcgcagcaagaggagccggctgctccacagcgactacatgaacatgacacccagaaggcctggccccaccaggaaacattatcagccctatgctccaccccgggactttgctgcttatagaagcagggtcaagttcagcagatctgcagatgccccagcctaccagcaggggcagaaccagctgtacaacgagctgaacctgggcaggagagaagaatatgacgtgttggacaagagaagaggaagagatcctgagatgggcgggaagcccaggaggaagaacccccaggagggcctgtataatgaactgcagaaagataaaatggccgaggcctattctgaaattggcatgaagggggagaggcggcgggggaaaggccatgatggactctaccaaggactgagcactgccacaaaggacacctatgatgctctgcacatgcaggccctgcctccaagatga

SEQ ID NO: 11 - DNA, 1977 bp, synthetic construct
  source 1..1977: mol_type = other DNA
  misc_feature 1..1977: note = Nucleotide sequence of anti-hCD19 CAR variant 3
atggatatccagatgacccagaccacatcttctctttctgccagcctgggggacagagtgacaatttcttgcagggcctcccaggacatctccaagtacctgaattggtaccagcaaaaaccagatggaacagtcaagctcctcatctaccacacatcaagacttcattctggcgtgcccagccgcttcagtgggagcggctcaggcacagattattctctcaccatttccaacctggagcaggaggacattgccacttacttctgccagcagggcaacacactgccctacacctttggaggtggcaccaaattggaaatcactggaagcacctctggatctgggaagccaggaagtggtgaaggctccaccaagggcgaggtgaaactgcaagaaagtggccctggcttggtggctccttctcagagcttgtctgtcacctgcacagtgtctggtgtgagcctgccagactatggggtgtcctggatccggcagccaccaagaaaaggactggagtggctgggagtgatctggggcagtgagaccacctactacaacagtgccctgaaatctcggctgactattatcaaggacaacagcaagagccaggtcttcctcaagatgaattcactgcagactgatgacacagccatctattactgcgccaagcactactattatggaggcagctacgccatggactactggggacaaggaacttctgtgactgtcagctctgagagcaaatatggcccaccctgtcctccctgccccatgttttgggtgctggtggtggttggtggagtcctggcctgctacagcctcctggtcactgtggccttcatcatcttctgggtcaaaagaggcaggaagaaacttctctacatctttaaacagcccttcatgagaccagtgcagacaacacaagaggaagatggctgcagctgcagatttccagaagaagaggaggggggctgtgagctgcgggtgaagttcagcaggtctgcagatgccccagcctatcagcagggacagaaccagctgtacaatgaactgaacctcggaagaagagaagaatatgacgtgttggacaagaggaggggcagagaccctgagatgggcggcaagccccggaggaaaaatcctcaggaagggctgtataatgagctccagaaggataaaatggccgaggcctacagtgaaattggcatgaagggagaaagaaggagaggaaaaggccatgatgggctctatcaaggcctcagcacagcaacaaaagacacctatgatgctctgcacatgcaggctcttcctccaaggctggaaggaggaggggagggccggggctctttgctgacctgtggagatgtggaggagaatccaggccccaggatgctgcttcttgtgaccagccttctgctgtgtgaacttccccaccctgcctttctgctgatccccagaaaggtgtgcaatggcatcggcattggagagttcaaagattctctgagcatcaatgccaccaacattaaacacttcaagaactgcacctccatcagtggagacctccacatcctgcctgtggcatttagaggtgacagcttcacccacacccctcctctggacccccaggagctggatattctgaagaccgtgaaggagatcacaggcttcctgctcattcaggcctggccagagaacaggacagacctgcatgcttttgaaaatttggagattattcgaggccgcaccaagcagcacggccagttctccctggctgtggtctccctcaacatcaccagtctgggcctgcgctccttgaaagaaatatctgatggtgatgtcatcatttcaggaaacaagaatctgtgctatgccaacaccatcaactggaagaagctgtttgggacctcagggcagaaaacaaagatcatcagcaacagaggggaaaactcctgcaaggccacaggtcaggtgtag

SEQ ID NO: 12 - DNA, 2466 bp, synthetic construct
  source 1..2466: mol_type = other DNA
  misc_feature 1..2466: note = Nucleotide sequence of anti-hCD19 CAR variant 4
atggatatccagatgacccagacaacatcaagtctgtctgccagccttggagacagagtgaccatctcttgcagggcctcccaggacatctccaaatacctgaattggtaccagcaaaaaccagatggcactgtcaaactcctcatctaccacacctccaggcttcattctggagtgcccagcagattttctggctctgggagcggcacagattattctctcaccatttccaacttggagcaggaagatattgctacttacttctgccagcagggaaatacactgccctacacctttggaggaggcaccaaactggagatcacaggatccaccagtggctcagggaagccaggaagtggtgagggaagcaccaagggcgaggtgaagctgcaagaaagtggacctggccttgtggctccttctcagagcttgtctgtcacctgcaccgtgtcaggagtttctcttccagactatggcgtgtcctggatccggcagcctccaaggaaaggactggagtggctgggtgtgatctggggcagtgagaccacctactacaacagtgccctgaaaagccgcctgacaattatcaaggacaattccaagagccaggtcttcctcaagatgaacagcctgcagactgatgacacagccatctattactgcgccaagcactactattatgggggctcctatgcaatggactactggggacaaggaacatctgtgacagtgtcttcagagagcaagtacggcccaccctgccctccttgccccatgttttgggtgctggttgtggtgggaggggtcctggcctgctacagcctgttggtgactgtggccttcatcatcttctgggtcaagaggggcaggaagaaacttttgtacatctttaaacagccctttatgagaccagtgcagaccacacaagaggaggacggctgcagctgccgcttcccagaagaagaggagggtggctgtgagctgcgggtgaagttcagcaggtctgcagatgcccctgcctatcagcagggccagaaccagctgtacaatgaactgaacctgggaaggagagaggaatatgacgtgttggacaaaagaagaggaagagaccccgagatgggcggcaagccccgccggaagaacccccaggaggggctctataatgagctccagaaagataaaatggccgaggcctacagtgaaataggaatgaagggagaaagaaggaggggaaaaggccatgatggactctaccaaggcctgagcacagcaacaaaagacacctatgatgcactgcacatgcaggctttgccacctcggctggaaggtggaggtgaaggccgcggcagcctcctgacctgtggagatgtggaagaaaatcctggacccaggatgctgctgctggtcacttctttgcttctctgtgaacttcctcacccagcctttctgctgatccccagaaaagtctgcaatggaattggcattggagagttcaaggattctctgtccatcaatgccaccaacattaaacacttcaaaaattgtaccagcatcagtggggacctccacatcctgcctgtggcatttagaggtgacagcttcacccacacaccaccactggatcctcaggagctggacattctgaagacagtaaaagaaatcactggcttcctgctcattcaagcctggccagagaacaggacagacctgcatgcttttgaaaatcttgaaattattcgaggcagaacaaagcagcacgggcagttctccctggctgtggtctccctcaacatcacctcactggggctgcgctccttgaaggagatttctgatggtgatgtcatcatttcaggcaacaagaatctgtgctatgccaacacaatcaactggaagaagctctttgggacctctggtcagaagaccaagatcatcagcaacagaggggaaaactcctgcaaggccactggacaggtgtgtcatgccctctgctccccggagggctgctgggggcctgagccccgggactgtgtcagctgtagaaatgtgagccggggccgggagtgtgtggacaagtgcaacctcttagaaggggagccaagagaatttgtggagaattctgagtgcatccagtgccatcctgaatgtctgccacaggccatgaatattacatgcactgggagaggccctgacaactgcattcagtgtgcccactatattgatgggccccactgcgtgaaaacatgtcctgctggggtgatgggagagaacaacaccctggtgtggaaatatgctgatgctggccacgtgtgccacctgtgtcaccccaactgtacatatggatgcacagggccaggcttggaaggatgtcccaccaatggccccaagattccttccattgccacaggcatggtgggcgccttgctgcttcttctggtggttgctctgggcatcggcctcttcatgtga

SEQ ID NO: 13 - DNA, 1401 bp, synthetic construct
  source 1..1401: mol_type = other DNA
  misc_feature 1..1401: note = Nucleotide sequence of anti-hCD19 CAR variant 5
atggacatccagatgacccagaccaccagcagcctgagcgccagcctgggcgacagagtgaccatcagctgcagagccagccaggacatcagcaagtacctgaactggtaccagcagaagcccgacggcaccgtgaagctgctgatctaccacaccagcagactgcacagcggcgtgcccagcagattcagcggcagcggcagcggcaccgactacagcctgaccatcagcaacctggagcaggaggacatcgccacctacttctgccagcagggcaacaccctgccctacaccttcggcggcggcaccaagctggagatcaccggcggcggcggcagcggcggcggcggcagcggcggcggcggcagcgaggtgaagctgcaggagagcggccccggcctggtggcccccagccagagcctgagcgtgacctgcaccgtgagcggcgtgagcctgcccgactacggcgtgagctggatcagacagccccccagaaagggcctggagtggctgggcgtgatctggggcagcgagaccacctactacaacagcgccctgaagagcagactgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctacaagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagatga

SEQ ID NO: 14 - DNA, 1407 bp, synthetic construct
  source 1..1407: mol_type = other DNA
  misc_feature 1..1407: note = Nucleotide sequence of anti-hCD19 CAR variant 6
atggatattcaaatgactcaaaccaccagcagcctctcggcgtcgctgggggaccgtgtcaccatctcctgccgggccagccaagatatttcaaaatatttaaattggtatcaacaaaaaccagatggaactgtaaaattattaatatatcacaccagccgcctgcacagtggtgttcccagccgcttcagtggaagtggaagtggaacagactacagcctcaccatttcaaatttagaacaagaagatattgccacctacttctgccaacaaggaaacacgctgccctacacgtttggtggtggcaccaaattagaaataactggcagcacctcgggctcggggaaaccaggaagtggagaaggcagcaccaaaggagaagtaaaattacaagaaagtgggccgggcctggtggcgcccagccaaagtttatctgtcacctgcactgtttctggtgtttctttaccagattatggtgtttcctggatccgccagccgccgaggaaaggattagaatggcttggtgtcatctggggctcggagaccacctactacaactcggcgctgaagagccgcctcaccataataaaagataattcaaaatcacaagtatttttaaaaatgaattcattacaaacagatgacacggccatctactactgtgcaaaacattattattatggtggctcctatgccatggactactggggacaaggaacttcagtaactgtttcctcggcggcggccattgaagtaatgtacccgccgccgtatttagataatgaaaaatcaaatggcaccatcattcatgtaaaaggaaaacatctctgcccctcgccgctcttcccggggcccagcaaacccttctgggttttagttgttgttggtggtgttttggcctgctacagtttattagtaactgtggccttcatcatcttctgggtccgcagcaaaagaagtcgtttattacattcagactacatgaacatgacgccgcggcggccggggcccaccaggaaacattatcagccctatgcgccgccgcgggactttgcagcctaccgcagccgtgtaaaattcagccgctcggcggatgccccggcctaccaacaaggacaaaatcaattatataatgaattaaatttaggaagaagagaagaatatgatgttttagataaaagaagaggaagagatcctgaaatgggaggaaaacccaggaggaaaaatcctcaagaaggtttatataatgaattacaaaaagataaaatggcggaggcctacagtgaaataggaatgaaaggagaaagaagaagaggaaaaggacatgatggtttatatcaaggcctctccacggccaccaaggacacctatgatgccctgcacatgcaggcgctgccgccgcggtga

SEQ ID NO: 15 - DNA, 1422 bp, synthetic construct
  source 1..1422: mol_type = other DNA
  misc_feature 1..1422: note = Nucleotide sequence of anti-BCMA CAR variant 1
atggatattgtgctcacccagagccctccttcactggcaatgtctctggggaagagagccactatttcttgccgggcctctgagagcgtgaccatcctgggcagccacctgatccactggtaccagcagaaacctgggcagccacccaccctcctcatccagctggccagcaatgtgcagacaggagtgcctgccaggttttctggaagtgggagcagaacagacttcaccctgaccattgatcctgtggaggaagatgatgtggccgtgtactactgtctccagtccaggaccatccccaggacatttggagggggcaccaagctggagatcaagggctccaccagcggctctgggaaaccaggctcaggagaaggaagcaccaaaggtcagattcagcttgttcagagtggaccagagctcaagaagccaggagagacagtgaagatctcctgcaaagccagtggctacaccttcacagattattctattaactgggtgaaaagggcccctggaaaaggactgaagtggatgggctggatcaatacagagaccagggagcctgcatatgcttatgactttagaggaagatttgctttcagcctggaaacatctgcctccactgcctacctgcagatcaacaacctgaaatatgaagacacagctacctacttctgtgccttggactattcctatgccatggactactggggccagggcacttctgtgactgtgtcctctgctgctgccaccaccacaccagctcctcggcccccaacacctgctcccacaattgcttcccagcctctgagcctcaggcctgaagcctgcaggccagcagctggaggagctgtgcacacaagagggctggattttgcctgtgacatctacatctgggcccccctggctgggacctgtggggtcctgctcctgtccctggtgatcaccttgtactgcaagaggggcaggaagaaactgctgtatatcttcaagcagcccttcatgagacctgtccagaccactcaggaggaggacggctgcagctgccgcttcccagaagaagaagagggcggctgtgaactgcgggtcaagttctccagatctgcagatgccccagcctatcagcaaggacagaaccagctctataatgagctgaacctcggccgccgggaggagtacgacgtgctggacaaaagaagaggccgggaccctgagatgggaggcaagccccggcggaagaacccccaggaaggcctgtacaatgaacttcagaaggacaagatggccgaggcctacagcgaaattggcatgaagggggagaggaggcggggcaaaggccatgatggactctaccaaggcctcagcacagccacaaaagatacctatgatgctctgcacatgcaggccctgcctccaagatga

SEQ ID NO: 16 - DNA, 1407 bp, synthetic construct
  source 1..1407: mol_type = other DNA
  misc_feature 1..1407: note = Nucleotide sequence of anti-BCMA CAR variant 2
atgcaagtgaaactggaggagtccggtggtggcttggtgcaagctggaagatcactgcgcctcagctgtgcagccagtgagcacaccttcagcagccacgtgatgggctggtttaggcaggctcctgggaaagaaagagagtctgtggccgtgattggctggagggacatttctacctcctatgcagacagcgtgaagggccgcttcaccatcagccgggacaacgccaagaaaacactttacctgcaaatgaacagcctcaagccagaggacacagcagtgtactattgtgccgcgcggaggattgatgcagctgactttgattcctggggacagggcacccaggtcaccgtgtcttctggaggaggtgggtcagaggtgcagctggtggagagtggtggagggctggttcaggcaggcggcagcctgcggctgtcctgcgccgcctctgggaggacctttaccatgggatggttccggcaggccccagggaaggagcgggagtttgtggctgccatctccctctctcccacactggcctattatgctgagagcgtcaaaggaaggttcacaatttcaagagacaatgccaaaaatacagtggtgctccagatgaattctctgaagcctgaagacaccgccctctactactgtgctgcagatagaaaatctgtcatgagcatccggccagactactggggccagggaacacaggtgactgtgtcctccaccagcaccaccacacctgcccctcggcccccaacaccagctcccaccattgccagccagcctctgagtctgaggcccgaggcctgcaggcctgctgctggcggggctgtgcacacacggggcctggacttcgcctgtgacatctacatctgggcccccctggctgggacctgtggggtcctgctgctctccctggtcatcaccctgtactgcaagaggggcaggaagaagctgctttatatcttcaagcagcccttcatgagacctgtccagaccactcaggaagaagatggctgcagctgccgctttccagaagaggaggaaggaggctgtgagctgagagtcaagttctccaggtctgctgatgctccagcctaccagcaggggcagaaccagctctacaatgagctcaacctgggccggagagaagaatatgacgtgctggataaaagaagaggcagagatcctgagatggggggcaagccccggcggaagaacccccaggagggcctctataatgaactgcagaaggacaagatggccgaggcttattctgagatcggcatgaagggggagaggaggagaggaaaaggccatgatggactgtatcagggtctgagcacagccaccaaagatacctatgatgccctgcacatgcaggccttgccacccaggtga

SEQ ID NO: 17 - DNA, 747 bp, synthetic construct
  source 1..747: mol_type = other DNA
  misc_feature 1..747: note = Nucleotide sequence of CVB3-IRES
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatgtttggggaaacccaaacatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagca

SEQ ID NO: 18 - protein, 21 aa, synthetic construct
  REGION 1..21: note = Amino acid sequence of CD8a signal peptide
MALPVTALLLPLALLLHAARP

SEQ ID NO: 19 - protein, 22 aa, synthetic construct
  REGION 1..22: note = Amino acid sequence of CSF2RA signal peptide
MLLLVTSLLLCELPHPAFLLIP

SEQ ID NO: 20 - DNA, 66 bp, synthetic construct
  source 1..66: mol_type = other DNA
  misc_feature 1..66: note = Nucleotide sequence of CSF2RA signal peptide
atgctgctgttggtgacttctctcctcctgtgtgagctgccccaccctgcctttctgctcatccca

SEQ ID NO: 21 - protein, 112 aa, synthetic construct
  REGION 1..112: note = Amino acid sequence of CD3 Zeta chain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - DNA, 336 bp, synthetic construct
  source 1..336: mol_type = other DNA
  misc_feature 1..336: note = Nucleotide sequence of CD3 Zeta chain
agggtcaagttctccagatctgcagatgccccagcctaccagcaggggcagaaccagctgtataatgaactgaacctgggccgccgggaggagtatgacgtgttggacaagagaagaggaagagatcctgagatgggcgggaagcccaggaggaagaacccccaggagggcctctacaacgagctccagaaagataaaatggccgaggcctattctgagatcggcatgaagggggagcggcgccgcgggaaaggccatgatggcctgtaccaaggactgagcactgccacaaaagacacctatgatgctctgcacatgcaggccctgccaccaagg

SEQ ID NO: 23 - protein, 107 aa, synthetic construct
  REGION 1..107: note = Amino acid sequence of CD28
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 24 - DNA, 321 bp, synthetic construct
  source 1..321: mol_type = other DNA
  misc_feature 1..321: note = Nucleotide sequence of CD28
attgaagtgatgtaccccccaccctacctggacaatgagaagagcaatggaacaatcattcatgtgaagggcaagcatctgtgccccagtcccctcttcccaggcccctccaaaccattttgggtcctggtggtggtcgggggcgtgctggcctgctacagcttgctggtcactgtggccttcatcatcttctgggtgcgcagcaagaggagccggctgctgcattctgattacatgaacatgacaccccggcggcctgggcccaccaggaaacattatcagccctatgctcctcctcgggactttgctgcttatagaagc

SEQ ID NO: 25 - protein, 248 aa, synthetic construct
  REGION 1..248: note = Amino acid sequence of anti-hCD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAA

SEQ ID NO: 26 - DNA, 744 bp, synthetic construct
  source 1..744: mol_type = other DNA
  misc_feature 1..744: note = Nucleotide sequence of anti-hCD19 scFv
gacatccagatgacccagaccaccagcagcctgtctgcctccctgggggacagagtgaccatctcctgccgggccagccaggacatttccaagtacctcaactggtaccagcagaagccagatggaactgtcaaacttctgatctaccacacctcccggctccacagcggggtgcccagccgcttcagcggctctggcagtgggacagactattccctcaccatcagcaacttggagcaggaagatattgccacctacttctgccagcaaggcaacaccctgccttacacctttggaggaggcaccaagctggaaattacaggctccaccagtggatctggaaaacctggctcaggagaaggaagcaccaaaggagaggtgaaactgcaagaaagtggccctggacttgtggctccatcccagagcctcagcgtcacctgcaccgtgtctggggtcagccttcctgattatggagtgtcctggatccggcagcctccaagaaaagggctggagtggctgggtgtgatctggggaagtgagaccacttactacaattcagccctcaagtccaggctgaccattatcaaggacaacagcaaatctcaggtgttcctgaagatgaattctctgcagacagatgacacagccatctattactgcgccaagcactactattatggcggcagctatgccatggactactggggccagggcacatctgtgacagtgagctctgctgccgcc

SEQ ID NO: 27 - DNA, 234 bp, synthetic construct
  source 1..234: mol_type = other DNA
  misc_feature 1..234: note = Nucleotide sequence of 5' Group I intron permuted-intron-exon homology arms
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagcc

SEQ ID NO: 28 - DNA, 187 bp, synthetic construct
  source 1..187: mol_type = other DNA
  misc_feature 1..187: note = Nucleotide sequence of 3' Group I intron permuted-intron-exon homology arms
ggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctag

SEQ ID NO: 29 - protein, 475 aa, synthetic construct
  REGION 1..475: note = Amino acid sequence of anti-mCD19 CAR
MGVPTQLLGLLLLWITDAICDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKGGGGSGGGGSGGGGSEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 30 - DNA, 1425 bp, synthetic construct
  source 1..1425: mol_type = other DNA
  misc_feature 1..1425: note = Nucleotide sequence of anti-mCD19 CAR
atgggagtgcccacccaactgctgggactcctgttgctgtggatcacagatgccatctgtgacattcagatgacccagtccccagccagcctctccaccagcctgggggagaccgtgaccatccagtgccaggcctcagaagacatctacagtgggctggcctggtaccaacagaagccaggaaagagccctcagctgctcatttatggagccagtgacctgcaggatggggtgcctagccgcttctctggcagcggcagtggcactcagtactccctgaagatcacctccatgcagacagaagatgaaggagtctacttctgtcagcaaggcctgacataccccaggacctttggaggtgggaccaaactggagctgaaaggcgggggaggctctgggggtggtggctccggtggaggaggctcagaggtgcagcttcagcagagtggagcagaactggtgcggcctggcacctctgtgaagctcagctgcaaggtgtctggagacaccattaccttctactacatgcacttcgtgaaacagagacctggacagggcttggagtggattggaagaattgacccagaggatgagagcaccaagtacagcgagaagttcaagaacaaggccactctgactgctgacacgtccagcaacacagcttacctcaagctgtcctctctgaccagtgaggacactgccacctacttttgcatctatgggggctactatttcgactactggggccagggtgtgatggtgacagtgtcctccattgagttcatgtacccacctccttacctggacaatgaaagaagcaatggaacaatcatccacatcaaggagaaacacctgtgccacacacagagcagccccaagcttttctgggccctggttgtggtggctggtgtcctgttctgctatggccttctggtcactgtggccctctgtgtgatctggaccaacagcagaagaaacagaggagggcagtctgactatatgaacatgacacccagaaggcctgggctcaccaggaagccctaccagccctatgcccctgccagagacttcgctgcctacaggccccgggccaagttttccaggtctgcagagacagcagccaacctccaggaccccaaccagctctttaatgagctcaacctgggccggcgggaggagtttgacgtgctggaaaagaagagagccagggaccctgagatgggcggcaagcagcagaggcggaggaacccccaggagggcgtgtacaacgccttgcagaaagacaagatggctgaggcctattctgagatcggcacaaaaggagagaggaggagagggaagggccatgatggcctcttccagggactgagcacagccacaaaggacacatttgatgctctgcacatgcagaccctggctcctcgg

SEQ ID NO: 31 - protein, 761 aa, synthetic construct
  REGION 1..761: note = Amino acid sequence of anti-CD20/CD19 bispecific CAR
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - DNA, 2286 bp, synthetic construct
  source 1..2286: mol_type = other DNA
  misc_feature 1..2286: note = Nucleotide sequence of anti-CD20/CD19 bispeccific CAR
atgctgcttctggtcacatctttgctgctgtgtgaacttccccaccccgccttcctcctgatcccagaggtgcagctccagcagagtggagcagagctggtaaaacctggggcctctgtgaagatgtcctgcaaggccagtggctacaccttcacctcttacaacatgcactgggtgaagcagactccagggcagggtctggagtggattggagccatttatccaggaaatggagacaccagctataaccagaagttcaagggaaaagccacgctgacagctgacaaatcttcctccactgcctacatgcaactgagctccctcacatcagaggacagtgctgattattattgtgctcgcagcaactactatggcagcagctactggttctttgatgtgtggggagctggaacaacagtgacagtcagcagtgggggcggcggcagcggaggcggcggatctggagggggagggagtgatattgtgctcactcagtccccagccatcctgtctgccagccctggagagaaggtgaccatgacctgccgcgcttcctcttcagtgaactatatggactggtatcaaaagaaaccaggctccagccccaagccctggatctatgcaacaagcaatctggcctcaggagtgcctgccaggttctccgggtccggctcgggcacttcctacagcctcaccatctccagagtggaggctgaggatgcagccacctattactgccagcagtggagcttcaacccacccacctttgggggtgggaccaagctggaaatcaaaggaggaggtggcagtggtggcggtggaagtggaggtggtggcagcggtggtgggggctcaggtggaggaggatcagatatccagatgacacagaccacttcttctctgtcagcctcgctgggggacagagtcaccatttcttgccgggcctcccaggacatcagcaagtacctgaactggtaccagcagaagccagatggaactgtcaaactcctcatctaccacacctccaggctgcattctggtgtgcccagccgcttcagcggctctgggagcgggacagactacagtctgactatttccaacttggagcaggaggacattgccacttacttctgccaacagggcaacaccctgccctacacatttggaggcggcacaaaactggagatcacaggaagcacctcagggtcaggcaagcctggttctggagaaggatccaccaaaggggaagtgaaacttcaagaaagtggccctggcctggtggccccttcacagagcctttctgtcacttgcaccgtgtctggcgtgtcactgccagactatggggtgtcctggatacggcagcctcccaggaaaggcttggaatggctgggagtcatctgggggtctgagaccacctactacaacagtgccctcaagtcccggctgaccatcatcaaggacaactccaagagccaggtgttcctgaagatgaacagcctgcaaacagatgacacagcaatttactactgtgccaagcactactactatggaggctcctatgccatggattactggggccagggaaccagtgtgactgtgagctctgctgctgccaccacaactcctgctcctcggcccccaacaccagcacccacaattgcttctcagccactgtccctgaggccagaagcctgcaggcctgcagctgggggggctgtgcacacaagaggactggattttgcctgtgacatctacatttgggcccccctggctggcacctgtggggtcctgctgctctccttggtgatcaccctctactgcaaaagaggcaggaagaagctcctttacatcttcaaacagcccttcatgagacctgtccagacaacccaggaagaagatggctgcagctgcagatttcctgaagaggaggaggggggctgtgagctgcgggtcaagttttctagatctgcagatgccccagcttaccagcaaggacagaatcagctgtacaatgagctcaacctgggccgccgggaggaatatgacgtgctggacaagaggcggggccgggaccctgagatgggcgggaaaccccggcggaagaacccccaggagggcctctataatgaactgcagaaagataaaatggccgaggcctattctgaaattggcatgaagggggagaggagaagagggaagggccatgatgggctgtatcaggggctcagcacagccacaaaggatacatatgatgctctgcacatgcaggccctgcctccaagatga

SEQ ID NO: 33 - protein, 245 aa, synthetic construct
  REGION 1..245: note = Amino acid sequence of anti-hCD19 CAR variant 2 binding scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGGSGGLITRLWCKLDSPASTKWLGVIWGSETTYYNSALKSRLTIIKDNSKSQVFLKMNSLQTDDTAIYYCAKHYYYGGSYAMDYWGQGTSVTVSS

SEQ ID NO: 34 - DNA, 735 bp, synthetic construct
  source 1..735: mol_type = other DNA
  misc_feature 1..735: note = Nucleotide sequence of anti-hCD19 CAR variant 2 binding scFv
gacatccagatgacccagaccaccagctccctgtctgcctctctgggggacagagtcaccatctcctgccgggccagccaggacatttccaagtacctcaactggtaccagcagaagccagatggcactgtcaaactcctgatctaccacacctccaggctgcattctggagtgcccagccgcttcagtgggagcggctctggcacagactattccctcaccatcagcaacttggagcaggaagatattgccacctacttctgccagcaaggcaacaccctgccctacacctttggaggagggaccaaactggagatcacaggcagcacctcaggctcaggaaaacctggaagtggagaaggaagcacaaaaggagaggtgaaacttcaagaaagtggccctgggctggtggctccttcccagagcctcagcgtgacctgcaccgtgtctgggggcagtggtggcctcatcacccgcctgtggtgcaagctggattctcctgcctccaccaagtggctgggagtcatctggggatctgagaccacttactacaattcagccctcaagtccagactgaccattattaaagacaacagcaaatctcaggtgttcctgaagatgaattctctccagacagatgacacagccatctattactgcgccaagcactactattatggaggctcctatgccatggattactggggccagggcacctctgtgacagtgtcctca

SEQ ID NO: 35 - protein, 242 aa, synthetic construct
  REGION 1..242: note = Amino acid sequence of anti-hCD19 CAR variant 5 binding scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 36 - DNA, 726 bp, synthetic construct
  source 1..726: mol_type = other DNA
  misc_feature 1..726: note = Nucleotide sequence of anti-hCD19 CAR variant 5 binding scFv
gacatccagatgacccagaccaccagcagcctgagcgccagcctgggcgacagagtgaccatcagctgcagagccagccaggacatcagcaagtacctgaactggtaccagcagaagcccgacggcaccgtgaagctgctgatctaccacaccagcagactgcacagcggcgtgcccagcagattcagcggcagcggcagcggcaccgactacagcctgaccatcagcaacctggagcaggaggacatcgccacctacttctgccagcagggcaacaccctgccctacaccttcggcggcggcaccaagctggagatcaccggcggcggcggcagcggcggcggcggcagcggcggcggcggcagcgaggtgaagctgcaggagagcggccccggcctggtggcccccagccagagcctgagcgtgacctgcaccgtgagcggcgtgagcctgcccgactacggcgtgagctggatcagacagccccccagaaagggcctggagtggctgggcgtgatctggggcagcgagaccacctactacaacagcgccctgaagagcagactgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 37 - protein, 246 aa, synthetic construct
  REGION 1..246: note = Amino acid sequence of anti-BCMA CAR variant 1 binding scFv
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 38 - DNA, 738 bp, synthetic construct
  source 1..738: mol_type = other DNA
  misc_feature 1..738: note = Nucleotide sequence of anti-BCMA CAR variant 1 binding scFv
gatattgtgctcacccagagccctccttcactggcaatgtctctggggaagagagccactatttcttgccgggcctctgagagcgtgaccatcctgggcagccacctgatccactggtaccagcagaaacctgggcagccacccaccctcctcatccagctggccagcaatgtgcagacaggagtgcctgccaggttttctggaagtgggagcagaacagacttcaccctgaccattgatcctgtggaggaagatgatgtggccgtgtactactgtctccagtccaggaccatccccaggacatttggagggggcaccaagctggagatcaagggctccaccagcggctctgggaaaccaggctcaggagaaggaagcaccaaaggtcagattcagcttgttcagagtggaccagagctcaagaagccaggagagacagtgaagatctcctgcaaagccagtggctacaccttcacagattattctattaactgggtgaaaagggcccctggaaaaggactgaagtggatgggctggatcaatacagagaccagggagcctgcatatgcttatgactttagaggaagatttgctttcagcctggaaacatctgcctccactgcctacctgcagatcaacaacctgaaatatgaagacacagctacctacttctgtgccttggactattcctatgccatggactactggggccagggcacttctgtgactgtgtcctct

SEQ ID NO: 39 - protein, 242 aa, synthetic construct
  REGION 1..242: note = Amino acid sequence of anti-BCMA CAR variant 2 binding scFv
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSS

SEQ ID NO: 40 - DNA, 726 bp, synthetic construct
  source 1..726: mol_type = other DNA
  misc_feature 1..726: note = Nucleotide sequence of anti-BCMA CAR variant 2 binding scFv
caagtgaaactggaggagtccggtggtggcttggtgcaagctggaagatcactgcgcctcagctgtgcagccagtgagcacaccttcagcagccacgtgatgggctggtttaggcaggctcctgggaaagaaagagagtctgtggccgtgattggctggagggacatttctacctcctatgcagacagcgtgaagggccgcttcaccatcagccgggacaacgccaagaaaacactttacctgcaaatgaacagcctcaagccagaggacacagcagtgtactattgtgccgcgcggaggattgatgcagctgactttgattcctggggacagggcacccaggtcaccgtgtcttctggaggaggtgggtcagaggtgcagctggtggagagtggtggagggctggttcaggcaggcggcagcctgcggctgtcctgcgccgcctctgggaggacctttaccatgggatggttccggcaggccccagggaaggagcgggagtttgtggctgccatctccctctctcccacactggcctattatgctgagagcgtcaaaggaaggttcacaatttcaagagacaatgccaaaaatacagtggtgctccagatgaattctctgaagcctgaagacaccgccctctactactgtgctgcagatagaaaatctgtcatgagcatccggccagactactggggccagggaacacaggtgactgtgtcctcc

SEQ ID NO: 41 - protein, 243 aa, synthetic construct
  REGION 1..243: note = Amino acid sequence of Leu-16 scFv
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 42 - DNA, 729 bp, synthetic construct
  source 1..729: mol_type = other DNA
  misc_feature 1..729: note = Nucleotide sequence of Leu-16 scFv
gaggtgcagctccagcagagtggagcagagctggtaaaacctggggcctctgtgaagatgtcctgcaaggccagtggctacaccttcacctcttacaacatgcactgggtgaagcagactccagggcagggtctggagtggattggagccatttatccaggaaatggagacaccagctataaccagaagttcaagggaaaagccacgctgacagctgacaaatcttcctccactgcctacatgcaactgagctccctcacatcagaggacagtgctgattattattgtgctcgcagcaactactatggcagcagctactggttctttgatgtgtggggagctggaacaacagtgacagtcagcagtgggggcggcggcagcggaggcggcggatctggagggggagggagtgatattgtgctcactcagtccccagccatcctgtctgccagccctggagagaaggtgaccatgacctgccgcgcttcctcttcagtgaactatatggactggtatcaaaagaaaccaggctccagccccaagccctggatctatgcaacaagcaatctggcctcaggagtgcctgccaggttctccgggtccggctcgggcacttcctacagcctcaccatctccagagtggaggctgaggatgcagccacctattactgccagcagtggagcttcaacccacccacctttgggggtgggaccaagctggaaatcaaa

SEQ ID NO: 43 - protein, 238 aa, synthetic construct
  REGION 1..238: note = Amino acid sequence of 1D3 scFv
DIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKGGGGSGGGGSGGGGSEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSS

SEQ ID NO: 44 - DNA, 714 bp, synthetic construct
  source 1..714: mol_type = other DNA
  misc_feature 1..714: note = Nucleotide sequence of 1D3 scFv
gacattcagatgacccagtccccagccagcctctccaccagcctgggggagaccgtgaccatccagtgccaggcctcagaagacatctacagtgggctggcctggtaccaacagaagccaggaaagagccctcagctgctcatttatggagccagtgacctgcaggatggggtgcctagccgcttctctggcagcggcagtggcactcagtactccctgaagatcacctccatgcagacagaagatgaaggagtctacttctgtcagcaaggcctgacataccccaggacctttggaggtgggaccaaactggagctgaaaggcgggggaggctctgggggtggtggctccggtggaggaggctcagaggtgcagcttcagcagagtggagcagaactggtgcggcctggcacctctgtgaagctcagctgcaaggtgtctggagacaccattaccttctactacatgcacttcgtgaaacagagacctggacagggcttggagtggattggaagaattgacccagaggatgagagcaccaagtacagcgagaagttcaagaacaaggccactctgactgctgacacgtccagcaacacagcttacctcaagctgtcctctctgaccagtgaggacactgccacctacttttgcatctatgggggctactatttcgactactggggccagggtgtgatggtgacagtgtcctcc

(19) United States
(12) Patent Application Publication
(10) Pub. No.: US 2026/0366031 A1
(43) Pub. Date: Nov. 19, 2026

(54) CIRCULAR RNA ENCODING CARS AND THE USE THEREOF

(71) Applicant: THERORNA SHANGHAI CO., LTD., Shanghai (CN)

(72) Inventors: Lu Gao, Shanghai (CN); Jing Zeng, Shanghai (CN); Chonghui Li, Shanghai (CN); Leshi Li, Shanghai (CN)

(21) Appl. No.: 19/721,577

(22) Filed: Jun. 26, 2026

Related U.S. Application Data
(63) Continuation of application No. PCT/CN2024/120871, filed on Sep. 24, 2024.

(30) Foreign Application Priority Data
Dec. 19, 2023 (WO) PCT/CN2023/143338
Jun. 12, 2024 (WO) PCT/CN2024/098800

(51) Int. Cl. C12N 15/63; A61K 48/00; A61P 35/02; C07K 14/705; C07K 16/28

(52) U.S. Cl. CPC C12N 15/63; A61K 48/0033; A61K 48/0058; A61K 48/0083; A61K 48/0091; A61P 35/02; C07K 14/7051; C07K 14/70517; C07K 14/70521; C07K 16/2803; C07K 16/2878; C07K 16/2887; C07K 2317/31; C07K 2317/35; C07K 2317/622; C07K 2319/02; C07K 2319/03; C12N 2310/532; C12N 2840/203

(57) ABSTRACT

The present invention provides a circular RNA encoding CARs and the use thereof to create immune cells that target specific diseases, e.g., lymphoma, multiple myeloma and leukemia and autoimmune diseases, such as systemic lupus erythematosus, lupus nephritis and myasthenia gravis.

This patent application is a continuation of International Application No. PCT/CN2024/120871, filed on Sep. 24, 2024, which claims priority to International Application No. PCT/CN2023/143338 filed on Dec. 29, 2023, and International Application No. PCT/CN2024/098800 filed on Jun. 12, 2024; the contents of each of the aforementioned applications are incorporated herein by reference in their entirety.

### INCORPORATION BY REFERENCE OF MATERIAL SUBMITTED ELECTRONICALLY

The Sequence Listing, which is a part of the present disclosure, is submitted concurrently with the specification as an XML file. The name of the XML file containing the Sequence Listing is “70820_SeqListing.xml”, which was created on Jun. 22, 2026, and is 88,350 bytes in size. The subject matter of the Sequence Listing is incorporated herein in its entirety by reference.

### TECHNICAL FIELD

The present invention relates to a circular RNA encoding CAR and related molecules and the use thereof for immunotherapy, such as treating cancers, hyperproliferative disease, fibrosis and autoimmune diseases.

### BACKGROUND OF THE INVENTION

Gene therapy offers a potential means to enhance immune recognition for specific disease targeting. Adoptive transfer of T cells engineered with chimeric antigen receptor (CAR) therapies have made tremendous breakthrough in cancer therapy against tumors of the B cell lineages. Thus far, six CAR T cell therapies have been approved by FDA targeting B cell leukemia, lymphoma, and multiple myeloma. However, the high cost and the complexity in manufacturing put this therapy beyond the reach of many patients who might benefit from. A solution could be to program T cell or other immune cells in vivo. Circular RNA can be particularly useful for in vivo protein expression therapies. The chimeric antigen receptors can be encoded by circular RNAs, then be in vivo delivered by LNP and expressed in immune cells. The application relates to circular RNA encoded chimeric T cell receptors, and methods of using it to facilitate immune response to the selected targets. Our circRNA CAR platform promise an effective, re-dosable and scalable off-the-shelf immune therapy without requiring lymphodepletion. To our surprise, a sustainable anti-tumor effect was observed with only two doses of CircRNA CAR in PBMC humanized mice. Significant anti-primary B cell activity was observed in non-human primates after the one dose. Furthermore, reduction of disease markers in a lupus mouse model was also achieved. These results demonstrated the potential of our circRNA CAR for clinical application in patients with cancer or autoimmune diseases.

### SUMMARY OF THE INVENTION

This invention relates to a novel circular RNA encoding chimeric antigen receptor (CAR) or a variant thereof which can recognize specific targets and treating diseases, through the therapeutic use of circular RNA.

In one aspect, the invention provides a composition comprising the circular RNA disclosed herein, wherein the composition comprises pharmaceutically acceptable excipients.

In one aspect, the invention provides a circular RNA comprising a regulatory element and an expression element described herein for pharmaceutical use.

In one aspect, the invention provides a method of immunotherapy in a subject comprising administering circular RNA to the subject. The immunotherapy is selected from CAR-T cell, CAR-NK cell, CAR-Macrophage cell or CAR-Treg therapies.

In one aspect, the immunotherapy is selected from treating cancer, hyperproliferative disease, fibrosis or autoimmune diseases. The circular RNA produces durable tumor control with almost completely tumor cells eradication and lasted for a long time after stopping the treatment.

In one aspect, the invention provides a linear RNA for manufacturing circular RNA disclosed herein.

In one aspect, the invention provides a DNA vector suitable for synthesizing the linear RNA and the circular RNA disclosed herein.

In one aspect, the invention provides a population of eukaryotic cells comprising a circular RNA disclosed herein.

In one aspect, the invention provides a method of manufacturing circular RNA disclosed herein, wherein the method comprises engagement of permuted intron-exon (PIE) system with group I introns or T4 RNA ligase 2 circulation.

### BRIEF DESCRIPTION OF THE DRAWINGS

FIG. 1 is the general structure of CAR-encoding circular RNAs.

FIGS. 2A-2E show the in vitro expression of anti-hCD19 CAR and anti-mCD19 CAR in the HEK293T and Jurkat cells transfected with corresponding circular RNAs. FIG. 2A-2C show the expression of CAR encoding circRNAs in HEK293T and Jurkat cells 12 hours post transfection.

FIGS. 2D-2E illustrate the duration of CAR expressions in vitro. CAR expression was evaluated by FCM from 6 h to 15 d post transfections.

FIGS. 3A-3F demonstrate the anti-tumor efficacy of administration of anti-hCD19 CAR circRNA on CD19+ B cell malignancy in humanized mice. FIG. 3A is a schematic illustration of the experimental overview for B cell leukemia treatment in humanized mouse. FIG. 3B is the representative image at indicated timepoints showing Nalm6 tumor flux in treated and control mice. FIG. 3C shows the total tumor flux value in treated and control mice. FIGS. 3D-3F show in vivo expression of anti-CD19 CAR in human T cell populations.

FIG. 4 shows anti-tumor activity at doses as low as 0.2 mg/kg.

FIGS. 5A-5I show the inhibitory effects of anti-hCD19 CAR circRNA on growth of malignant CD19+ B cell in humanized mice. Treatment of circRNA CAR encapsulated fabricated LNP (fLNP) efficiently delayed Nalm6 tumor growth in M-NSG mice (FIGS. 5A-5C). In vivo expression of CARs and human T cell development was examined by flow cytometry (FIGS. 5D-5I).

FIGS. 6A-6I show potent anti-tumor efficacy of in vivo CAR with manageable cytokine release in preclinical mouse model of B cell leukemia. FIG. 6A is the experimental design of anti-tumor efficacy study on A20-BALB/c mouse model. FIG. 6B shows anti-mCD19 CAR circRNA treatment delayed tumor progression in A20 syngeneic model. FIG. 6C shows the body weight change of administration of anti-mCD19 CAR circRNA and FIGS. 6D-6I show the serum cytokine levels 12 h after anti-mCD19 CAR circRNA injection.

FIGS. 7A-7F show the efficacy of anti-mCD19 CAR circRNA on lupus pathogenesis. FIG. 7A shows experimental overview for systemic lupus erythematosus treatment by circRNA-CAR in MRL-lpr mice. FIGS. 7B-7C show the serology analysis anti-dsDNA antibody and total mouse IgG. FIGS. 7D-7F show proteinuria and CREA detection results which indicate kidney injury.

FIGS. 8A-8C demonstrate circRNA-CAR induce potent B-cell depletion in non-human primates (NHPs). FIG. 8A depicts the NHP study design. FIG. 8B illustrates the CD20/CD19 bispecific CAR structure. B cells in the periphery blood were specifically depleted post intravenous injection of anti-CD20/19 bispecific CAR (FIG. 8C).

### DETAILED DESCRIPTION OF THE INVENTION

Unless otherwise defined below, all technical and scientific terms used herein have the same meanings as commonly understood by an ordinary skilled person in the art. References to techniques used herein are intended to refer to techniques that are generally understood in the art, including those obvious changes or equivalent replacements of the techniques for those skilled in the art. While it is believed that the following terms are well understood by those skilled in the art, the following definitions are set forth to better explain the invention.

### General Definition

As used herein, the terms “including”, “comprising”, “having”, “containing” or “comprising”, and other variants thereof, are inclusive or open, and do not exclude other unlisted elements or method steps. In some embodiments, the “including”, “comprising”, “having”, “containing” or “comprising” could be replaced with “consisting of” or “consists”.

As used herein, the terms “embodiment”, “disclosed herein” or “disclosure” are not meant to be limiting, but applies generally to any of the embodiments defined in the claims or described herein. These terms are used interchangeably herein.

As used herein, the terms “treat”, “treating”, “treatment” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of the circular RNA or the composition disclosed herein to a subject in need of such treatment. The treatment can be orientated symptomatically, for example, to suppress symptoms. It can be effected over a short period, be oriented over a medium term, or can be a long-term treatment, for example within the context of a maintenance therapy.

Throughout this disclosure, the terms “a” or “an” entity refers to one or more of that entity; for example, “a polynucleotide” is understood to represent one or more polynucleotides. As such, the terms “a” (or “an”), “one or more” and “at least one” can be used interchangeably herein.

The term “variant”, as used herein, refers to a peptide that differs from the recited peptide due to amino acid substitutions, deletions, insertions, and/or modifications. Variants can be produced using art-known mutagenesis techniques.

The terms “composition” or “pharmaceutical composition” refer to compositions comprising the circular RNA provided herein, along with e.g., pharmaceutically acceptable carriers, excipients, or diluents for administration to a subject in need of treatment.

The term “pharmaceutically acceptable” refers to compositions that are, within the scope of sound medical judgment, suitable for contact with the tissues of human beings and animals without excessive toxicity or other complications commensurate with a reasonable benefit/risk ratio.

An “effective amount” is that amount of a circular RNA provided herein, the administration of which to a subject, either in a single dose or as part of a series, is effective for treatment. This amount can be a fixed dose for all subjects being treated, or can vary depending upon the weight, health, and physical condition of the subject to be treated, the extent of weight loss or weight maintenance desired, the formulation of the circular RNA or the composition disclosed herein, a professional assessment of the medical situation, and other relevant factors.

The term “subject” is meant any subject, particularly a mammalian subject, in need of treatment with the circular RNA or the composition provided herein. Mammalian subjects include, but are not limited to, humans, dogs, cats, guinea pigs, rabbits, rats, mice, horses, cattle, bears, cows, apes, monkeys, orangutans, and chimpanzees, and so on. In one embodiment, the subject is a human subject.

### Circular RNA

As used herein, the terms “circRNA” or “circular polyribonucleotide” or “circular RNA” are used interchangeably and can refer to a polyribonucleotide that forms a circular structure through covalent or non-covalent bonds. When it comes to circular RNA, a skilled person would understand that a polynucleotide in a RNA refers to a polyribonucleotide.

In one aspect, the invention provides a circular RNA encoding chimeric antigen receptor (CAR) or a variant thereof.

In some embodiments, the circular RNA encoding chimeric antigen receptor (CAR), comprises a regulatory element and an expression element comprising a polynucleotide encoding chimeric antigen receptor (CAR); and
the chimeric antigen receptor (CAR) comprising an antigen binding domain, a transmembrane domain, a co-stimulatory domain, and an intracellular T cell signaling domain;
wherein each element is optionally connected by a polynucleotide encoding a linker.

In some embodiments, the light chain variable region and heavy chain variable region in the CAR are optionally connected by a linker, wherein the linker is selected or derived from GA, GS, GG, GGA, GGS, GGG, GGGA, GGGS, GGGG, GGGGA, GGGGG, GGGGA, GGGGS, GGGGG, GGAG, GGSG, AGGGG, SGGG, GAGA, GSGS, GAGAGA, GSGSGS, GAGAGAGA, GSGSGSGS, GAGAGAGAGA, GSGSGSGSGS, GAGAGAGAGAGA, GAGSGSGSGSGS, GGAGGA, GGSGGS, GGAGGAGGA, GGSGGSGGS, GGAGGAGGAGGA, GGSGGSGGSGGS, GGAGGGAG, GGSGGGSG, GGAGGGAGGGAG, GGSGGGSGGSG, GGGAGGGAGGGA, GGGSGGGSGGGS, (GGGGS)₃ or GSTSGSGKPGSGEGSTKG.

In some embodiments, the polynucleotide encoding CAR or a variant thereof has at least 50%, at least 60%, at least 70%, at least 80%, at least 90%, at least 95%, at least 98%, at least 99% or 100% identity with the sequence set forth as SEQ ID NO: 9, 10, 11, 12, 13, 14, 15, 16 or 30.

In some embodiments, the amino acid sequence or a variant thereof of the CAR encoded by the polynucleotide has at least 50%, at least 60%, at least 70%, at least 80%, at least 90%, at least 95%, at least 98%, at least 99% or 100% identity with the sequence set forth as SEQ ID NO: 1, 2, 3, 4, 5, 6, 7, 8 or 29.

In some embodiments, the circular RNA further comprises polynucleotide encoding a signal peptide, the signal peptide is selected from the group consisting of CD8a leader sequence has at least 50%, at least 60%, at least 70%, at least 80%, at least 90%, at least 95%, at least 98%, at least 99% or 100% identity with the sequence set forth as SEQ ID NO: 18, and colony stimulating factor 2 receptor subunit alpha (CSF2RA) signal peptide has at least 50%, at least 60%, at least 70%, at least 80%, at least 90%, at least 95%, at least 98%, at least 99% or 100% identity with the sequence set forth as SEQ ID NO: 19.

In some embodiments, wherein the antigen binding domain is one or more antigen binding domain(s) specific for CD19, CD20, BCMA, CLDN6, CLDN18.2, ERBB2 (HER2), PSMA, SLAMF3, SLAMF7, CD66c, MSLN, CD38, CD123, GPC3, EGFRvIII, CD171, MUC1 and/or GPRC5D; preferably is a single chain variable fragment (scFv) specific for CD19, CD20 and/or BCMA, more preferably is the amino acid sequence of scFv having at least 50%, at least 60%, at least 70%, at least 80%, at least 90%, at least 95%, at least 98%, at least 99% or 100% identity with the sequence set forth as SEQ ID NO: 25, 33, 35, 37, 39 or 41;
wherein the amino acid sequence of the scFv is encoded by a polynucleotide sequence having at least 50%, at least 60%, at least 70%, at least 80%, at least 90%, at least 95%, at least 98%, at least 99% or 100% identity with the sequence set forth as SEQ ID NO: 26, 34, 36, 38, 40 or 42.

In some embodiments, wherein the antigen binding domain is one or more antigen binding domain(s) specific for CD19, wherein the CD19 may be humanized CD19, partially humanized CD19, or murine CD19.

In some embodiments, wherein the antigen binding domain is one or more antigen binding domain(s) specific for CD20, wherein the CD20 may be humanized CD20, partially humanized CD20, or murine CD20.

In some embodiments, wherein the antigen binding domain is one or more antigen binding domain(s) specific for BCMA, wherein the BCMA may be humanized BCMA, partially humanized BCMA, or murine BCMA. In some embodiments, the co-stimulatory domain is selected from the group consisting of CD28, 4-1BB, OX40, CD27, CD30, ICOS, GITR, CD40, CD2, SLAM, and combinations thereof; preferably CD28 or 4-1BB; more preferably the co-stimulatory domain of CD28 has at least 50%, at least 60%, at least 70%, at least 80%, at least 90%, at least 95%, at least 98%, at least 99% or 100% identity with the sequence set forth as SEQ ID NO: 23.

In some embodiments, the transmembrane domain is selected from the group consisting of CD28 and CD8 transmembrane domain.

In some embodiments, the intracellular T cell signaling domain is CD3zeta signaling domain. preferably the CD3zeta signaling domain has at least 50%, at least 60%, at least 70%, at least 80%, at least 90%, at least 95%, at least 98%, at least 99% or 100% identity with the sequence set forth as SEQ ID NO: 21.

In some embodiments, the circular RNA of the present invention comprises a regulatory element and an expression element comprising a polynucleotide encoding a bispecific CAR specific for CD20 and CD19,
wherein the regulatory element comprises a polynucleotide encoding an internal ribosomal entry site (IRES) or a fragment thereof as defined in claim 9;
wherein the bispecific CAR comprises an antigen binding domain, a co-stimulatory domain disclosed herein, a transmembrane domain disclosed herein, and an intracellular T cell signaling domain disclosed herein;
wherein the antigen binding domain is an antigen binding domain specific for CD19 and CD20; preferably comprises single chain variable fragments (scFv); more preferably are FMC-63 scFv and Leu-16 scFv;
wherein the FMC-63 scFv is the amino acid sequence of scFv having at least 50%, at least 60%, at least 70%, at least 80%, at least 90%, at least 95%, at least 98%, at least 99% or 100% identity with the sequence set forth as the amino acid sequence selected from SEQ ID NO: 25, 33, or 35; wherein the Leu-16 scFv is the amino acid sequence of scFv having at least 50%, at least 60%, at least 70%, at least 80%, at least 90%, at least 95%, at least 98%, at least 99% or 100% identity with the sequence set forth as SEQ ID NO:41;
wherein each element is optionally connected by a polynucleotide encoding a linker.

In some embodiments, the polynucleotide encoding CAR or a variant thereof has at least 50%, at least 60%, at least 70%, at least 80%, at least 90%, at least 95%, at least 98%, at least 99% or 100% identity with the sequence set forth as SEQ ID NO: 32.

In some embodiments, wherein an amino acid sequence or a variant thereof of the CAR encoded by the polynucleotide has at least 50%, at least 60%, at least 70%, at least 80%, at least 90%, at least 95%, at least 98%, at least 99% or 100% identity with the sequence set forth as SEQ ID NO: 31.

In some embodiments, the circular RNA of the present invention comprises a regulatory element and an expression element comprising a polynucleotide encoding CAR,
wherein the regulatory element is CVB3 IRES having the sequence set forth as SEQ ID NO: 17;
wherein the CAR comprises an antigen binding domain, a transmembrane domain, a co-stimulatory domain, an intracellular T cell signaling domain and a signal peptide element;
wherein the antigen binding domain is specific for hCD19 or mCD19, wherein the antigen binding domain is the amino acid sequence of scFv having the sequence set forth as SEQ ID NO: 25, 33, 35, 37, 39 or 41;
wherein the co-stimulatory domain is the co-stimulatory domain of CD28 having the sequence set forth as SEQ ID NO: 23;
wherein the transmembrane domain is CD28 or CD8 transmembrane domain;
wherein the intracellular T cell signaling domain is CD3zeta signaling domain having the sequence set forth as SEQ ID NO: 21; and
wherein the signal peptide element is colony stimulating factor 2 receptor subunit alpha (CSF2RA) signal peptide having the sequence set forth as SEQ ID NO: 19;
wherein each element is connected by a polynucleotide encoding (GGGGS)₃.

### Regulatory Elements

A regulatory element may include a sequence that is located adjacent to an expression element that encodes an expression product. A regulatory element may be linked operatively to the adjacent sequence. A regulatory element may increase the amount of the product expressed as compared to the amount of the expressed product when no regulatory element exists. In addition, one regulatory element can increase the amount of products expressed for multiple expression sequences attached in tandem. Hence, one regulatory element can enhance the expression of one or more expression sequences.

In some embodiments, the regulatory element increases the translation production of circular RNA. In some embodiments, the regulatory element comprises an internal ribosomal entry site (IRES) or a fragment thereof.

A suitable IRES element to include in a circular polyribonucleotide comprises an RNA sequence capable of engaging a eukaryotic ribosome. In some embodiments, the IRES element is at least about 5 nt, at least about 8 nt, at least about 9 nt, at least about 10 nt, at least about 15 nt, at least about 20 nt, at least about 25 nt, at least about 30 nt, at least about 40 nt, at least about 50 nt, at least about 100 nt, at least about 200 nt, at least about 250 nt, at least about 350 nt, or at least about 500 nt.

In some embodiments, the IRES is selected from the group consisting of Coxsackievirus B3 (CVB3) IRES, Enterovirus 71 (EV71) IRES, encephalomyocarditis virus (EMCV) IRES, picornavirus (PV) IRES, hepatitis C virus (HCV) IRES, adenovirus (AdV) IRES, human papillomavirus type 31 (HPV31) IRES, human herpesvirus (HHV) IRES, Rous sarcoma virus (RSV) IRES, classical swine fever virus (CSFV) IRES, FGF9 IRES, SLC7A1 IRES, and RUNX1 IRES; preferably the IRES is CVB3 IRES or a variant thereof has at least 50%, at least 60%, at least 70%, at least 80%, at least 90%, at least 95%, at least 98%, at least 99% or 100% identity with the sequence set forth as SEQ ID NO: 17.

### Pharmaceutical Compositions

In one aspect, the invention provides a composition comprising the circular RNA of any one of the preceding embodiments, wherein the composition comprises pharmaceutically acceptable excipients.

In some embodiments, the composition comprises nanoparticle, for example, lipid nanoparticle.

In some embodiments, the circRNA is administered as naked circRNA, or as a pharmaceutical composition comprising a pharmaceutically acceptable excipient. In non-limiting examples, the pharmaceutically acceptable excipient is polyethyleneimine (PEI) or a lipid nanoparticle (LNP). Other examples of liposomes that can be used to administer the circRNA or the composition for administration include protamines, cationic nanoemulsions, modified dendrimer nanoparticles, protamine liposomes, cationic polymers, cationic polymer liposomes, polysaccharide particles, cationic lipid nanoparticles, cationic lipid-cholesterol nanoparticles, cationic lipid-cholesterol PEG nanoparticle, cationic lipid transfection reagents sold under the trademark LIPOFECTAMINE, nonliposomal transfection reagents sold under the trademark FUGENE, or any combination thereof can be used as the pharmaceutically acceptable excipient.

In some embodiments, the lipid nanoparticle comprises at least one of an ionizable lipid, a structural lipid, and a PEG-modified lipid; preferably the ionizable lipid is SM102 (CAS No. 2089251-47-6).

In some embodiments, the pharmaceutical compositions may optionally comprise one or more additional active substances, e.g. therapeutically and/or prophylactically active substances.

### Diseases

In one aspect, the invention provides a method for immunotherapy in a subject, comprising administering a therapeutically effective amount of the circular RNA or the composition disclosed herein to the subject.

In one aspect, the invention provides the use of the circular RNA or the composition disclosed herein in manufacture of a medicament for immunotherapy in a subject.

In one aspect, the invention provides a circular RNA or the composition disclosed herein, for use in immunotherapy in a subject.

In some embodiments, the immunotherapy is selected from CAR-T cell, CAR-NK cell, CAR-Macrophage cell or CAR-Treg therapies.

In some embodiments, the immunotherapy is selected from treating cancer, hyperproliferative disease, fibrosis or autoimmune diseases. The cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors.

In some embodiments, the cancer is selected from the group consisting of acute lymphocytic cancer, acute myeloid leukemia (AML), alveolar rhabdomyosarcoma, bladder cancer (e.g., bladder carcinoma), bone cancer, brain cancer (e.g., medulloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non small cell lung carcinoma and lung adenocarcinoma), lymphoma, mesothelioma, mastocytoma, melanoma, multiple myeloma, nasopharynx cancer, non-Hodgkin lymphoma, B-chronic lymphocytic leukemia, hairy cell leukemia, acute lymphocytic leukemia (ALL), and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, synovial sarcoma, gastric cancer, testicular cancer, thyroid cancer, and ureter cancer.

In some embodiments, the autoimmune disease is selected from systemic lupus erythematosus (SLE), lupus nephritis, myasthenia gravis, pemphigus vulgaris, or type I diabetes.

In some embodiments, the fibrosis is cardiac fibrosis.

### Linear RNA

In one aspect, the invention provides a linear RNA for manufacturing circular RNA disclosed herein.

In some embodiments, the linear RNA polynucleotide comprising a regulatory element comprises a polynucleotide encoding an internal ribosomal entry site (IRES) or a fragment thereof of the preceding embodiments, an expression element encoding a chimeric antigen receptor (CAR) polypeptide of any one of preceding embodiments, and at least one self-circularizing element;
wherein each element is optionally connected by a polynucleotide encoding a linker, wherein the linker is selected or derived from GA, GS, GG, GGA, GGS, GGG, GGGA, GGGS, GGGG, GGGGA, GGGGG, GGGGA, GGGGS, GGGGG, GGAG, GGSG, AGGGG, SGGG, GAGA, GSGS, GAGAGA, GSGSGS, GAGAGAGA, GSGSGSGS, GAGAGAGAGA, GSGSGSGSGS, GAGAGAGAGAGA, GAGSGSGSGSGS, GGAGGA, GGSGGS, GGAGGAGGA, GGSGGSGGS, GGAGGAGGAGGA, GGSGGSGGSGGS, GGAGGGAG, GGSGGGSG, GGAGGGAGGGAG, GGSGGGSGGSG, GGGAGGGAGGGA, GGGSGGGSGGGS or (GGGGS)₃.

In some embodiments, the linear RNA polynucleotide comprises, in the following order from 5′ to 3′:
optionally a 5′-self-circularizing element;
a regulatory element comprises a polynucleotide encoding an internal ribosomal entry site (IRES) or a fragment thereof disclosed herein;
optionally a signal peptide element disclosed herein;
an expression element comprising a polynucleotide encoding chimeric antigen receptor (CAR) of any one of the preceding embodiments;
optionally a stop codon;
optionally a 3′-self-circularizing element.

In some embodiments, the 5′-self-circularizing elements comprises a 5′-Group I intron fragment and an Exon2, preferably, a 5′ Group I intron permuted-intron-exon homology arms has at least 50%, at least 60%, at least 70%, at least 80%, at least 90%, at least 95%, at least 98%, at least 99% or 100% identity with the sequence set forth as SEQ ID NO: 27. In some embodiments, the 3′-self-circularizing elements comprises an Exon1 and 3′-Group I intron fragment; preferably, a nucleotide sequence of 3′ Group I intron permuted-intron-exon homology arms has at least 50%, at least 60%, at least 70%, at least 80%, at least 90%, at least 95%, at least 98%, at least 99% or 100% identity with the sequence set forth as SEQ ID NO: 28. In some embodiments, the 3′ group I intron fragment and the 5′ group I intron fragment are Anabaena group I intron fragments.

In some embodiments, the self-circularizing element is T4 RNA ligase 2 circulation, and the 5′ end of the linear RNA polynucleotide is phosphorylated, preferably monophosphate, the 3′ end of the linear RNA polynucleotide is OH. The T4 RNA ligase 2 catalyzes the formation of a phosphodiester bond between 5-phosphate (donor) and 3-hydroxyl (acceptor) end groups in RNA in an ATP-dependent reaction.

In some embodiments, the linear RNA polynucleotide comprises, in the following order from 5′ to 3′:
5′ Group I intron permuted-intron-exon homology arms having the sequence set forth as SEQ ID NO: 27;
a regulatory element comprises a polynucleotide encoding an internal ribosomal entry site (IRES) or a fragment thereof disclosed herein, wherein the IRES is CVB3 IRES having the sequence set forth as SEQ ID NO: 17;
a signal peptide element, wherein the signal peptide element is colony stimulating factor 2 receptor subunit alpha (CSF2RA) signal peptide having the sequence set forth as SEQ ID NO: 19;
an expression element comprising a polynucleotide encoding CAR), wherein the expression element comprising an antigen binding domain, a transmembrane domain, a co-stimulatory domain, an intracellular T cell signaling domain and a signal peptide element; wherein the antigen binding domain is specific for hCD19 or mCD19, wherein the antigen binding domain is the amino acid sequence of scFv having the sequence set forth as SEQ ID NO: 25, 33, 35, 37, 39 or 41; wherein the co-stimulatory domain is the co-stimulatory domain of CD28 having the sequence set forth as SEQ ID NO: 23; wherein the transmembrane domain is CD28 or CD8 transmembrane domain; wherein the intracellular T cell signaling domain is CD3zeta signaling domain having the sequence set forth as SEQ ID NO: 21; and wherein the signal peptide element is colony stimulating factor 2 receptor subunit alpha (CSF2RA) signal peptide having the sequence set forth as SEQ ID NO: 19;
a stop codon; and
3′ Group I intron permuted-intron-exon homology arms having the sequence set forth as SEQ ID NO: 28;
wherein each element is optionally connected by a polynucleotide encoding (GGGGS)₃.

### DNA Vector and Population of Eukaryotic Cells

In one aspect, the invention provides a DNA vector suitable for synthesizing the linear RNA and the circular RNA disclosed herein.

In one aspect, the invention provides a population of eukaryotic cells comprising a circular RNA disclosed herein.

In some embodiments, the population of eukaryotic cells express the CAR complex protein encoded by the circular RNA polynucleotide on its cell surface.

In some embodiments, the population of cells kills tumor cells more effectively or for longer than an equivalent population of eukaryotic cells comprising linear RNA encoding the same CAR

### Method of Manufacturing the Circular RNA

In one aspect, the invention provides a method of manufacturing circular RNA disclosed herein, wherein the method comprises engagement of permuted intron-exon (PIE) system with group I introns or T4 RNA ligase 2 circulation.

In some embodiments, the method of manufacturing the circular RNA of the invention comprises engagement of permuted intron-exon (PIE) system with group I introns.

In some embodiments, the method of manufacturing the circular RNA of the invention comprises engagement of T4 RNA ligase 2 circulation.

In some embodiments, the circular RNA is circularized from the linear RNA polynucleotide of the preceding embodiments.

### EXAMPLES

To make the objects and technical solutions of the present invention clearer, the present invention will be further described below in conjunction with specific examples. It should be understood that the examples are not intended to limit the scope of the invention. Further, specific experimental methods not mentioned in the following examples were carried out in accordance with a conventional experimental method.

The sequence numbers used in the invention are listed in table 1. The nucleotide sequence shown in the sequence listing can represent RNA and can be converted into a WIPO standard ST.26 sequence listing.

TABLE 1

| NO. | Description |
|---|---|
| SEQ ID NO: 1 | Amino acid sequence of anti-hCD19 CAR variant 1 |
| SEQ ID NO: 2 | Amino acid sequence of anti-hCD19 CAR variant 2 |
| SEQ ID NO: 3 | Amino acid sequence of anti-hCD19 CAR variant 3 |
| SEQ ID NO: 4 | Amino acid sequence of anti-hCD19 CAR variant 4 |
| SEQ ID NO: 5 | Amino acid sequence of anti-hCD19 CAR variant 5 |
| SEQ ID NO: 6 | Amino acid sequence of anti-hCD19 CAR variant 6 |
| SEQ ID NO: 7 | Amino acid sequence of anti-BCMA CAR variant 1 |
| SEQ ID NO: 8 | Amino acid sequence of anti-BCMA CAR variant 2 |
| SEQ ID NO: 9 | Nucleotide sequence of anti-hCD19 CAR variant 1 |
| SEQ ID NO: 10 | Nucleotide sequence of anti-hCD19 CAR variant 2 |
| SEQ ID NO: 11 | Nucleotide sequence of anti-hCD19 CAR variant 3 |
| SEQ ID NO: 12 | Nucleotide sequence of anti-hCD19 CAR variant 4 |
| SEQ ID NO: 13 | Nucleotide sequence of anti-hCD19 CAR variant 5 |
| SEQ ID NO: 14 | Nucleotide sequence of anti-hCD19 CAR variant 6 |
| SEQ ID NO: 15 | Nucleotide sequence of anti-BCMA CAR variant 1 |
| SEQ ID NO: 16 | Nucleotide sequence of anti-BCMA CAR variant 2 |
| SEQ ID NO: 17 | Nucleotide sequence of CVB3-IRES |
| SEQ ID NO: 18 | Amino acid sequence of CD8a signal peptide |
| SEQ ID NO: 19 | Amino acid sequence of CSF2RA signal peptide |
| SEQ ID NO: 20 | Nucleotide sequence of CSF2RA signal peptide |
| SEQ ID NO: 21 | Amino acid sequence of CD35 chain |
| SEQ ID NO: 22 | Nucleotide sequence of CD32 chain |
| SEQ ID NO: 23 | Amino acid sequence of CD28 |
| SEQ ID NO: 24 | Nucleotide sequence of CD28 |
| SEQ ID NO: 25 | Amino acid sequence of anti-CD19 scFv |
| SEQ ID NO: 26 | Nucleotide sequence of anti-CD19 scFv |
| SEQ ID NO: 27 | Nucleotide sequence of 5′ Group I intron permuted- |
| | intron-exon homology arms |
| SEQ ID NO: 28 | Nucleotide sequence of 3′ Group I intron permuted- |
| | intron-exon homology arms |
| SEQ ID NO: 29 | Amino acid sequence of anti-mCD19 CAR |
| SEQ ID NO: 30 | Nucleotide sequence of anti-mCD19 CAR |
| SEQ ID NO: 31 | Amino acid sequence of anti-CD20/CD19 bispecific |
| | CAR |
| SEQ ID NO: 32 | Nucleotide sequence of anti-CD20/CD19 bispeccific |
| | CAR |
| SEQ ID NO: 33 | Amino acid sequence of anti-hCD19 CAR variant |
| | 2 binding scFv |
| SEQ ID NO: 34 | Nucleotide sequence of anti-hCD19 CAR variant |
| | 2 binding scFv |
| SEQ ID NO: 35 | Amino acid sequence of anti-hCD19 CAR variant |
| | 5 binding scFv |
| SEQ ID NO: 36 | Nucleotide sequence of anti-hCD19 CAR variant |
| | 5 binding scFv |
| SEQ ID NO: 37 | Amino acid sequence of anti-BCMA CAR variant |
| | 1 binding scFv |
| SEQ ID NO: 38 | Nucleotide sequence of anti-BCMA CAR variant |
| | 1 binding scFv |
| SEQ ID NO: 39 | Amino acid sequence of anti-BCMA CAR variant |
| | 2 binding scFv |
| SEQ ID NO: 40 | Nucleotide sequence of anti-BCMA CAR variant |
| | 2 binding scFv |
| SEQ ID NO: 41 | Amino acid sequence of Leu-16 scFv |
| SEQ ID NO: 42 | Nucleotide sequence of Leu-16 scFv |
| SEQ ID NO: 43 | Amino acid sequence of 1D3 scFv |
| SEQ ID NO: 44 | Nucleotide sequence of 1D3 scFv |

### Example 1 Vector Construction

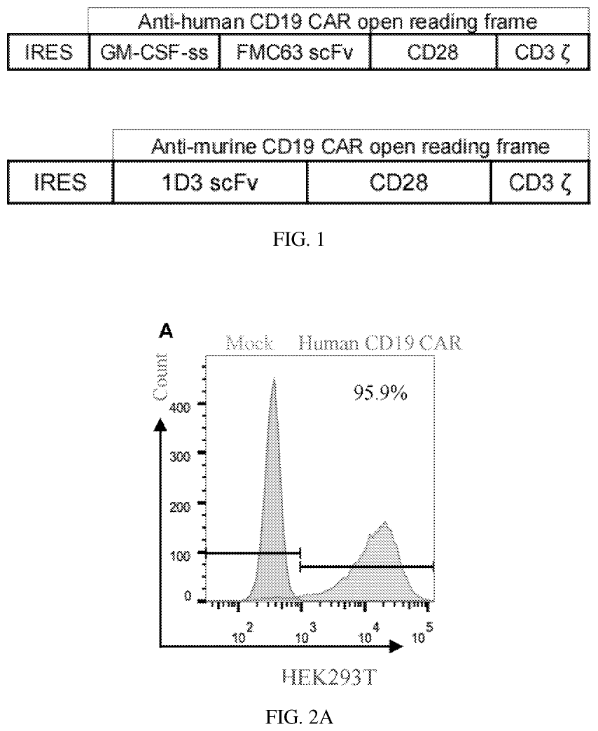

The open reading frame of circular RNA encoding anti-hCD19 CAR consists of three functional domains (FIG. 1 top), one is a single-chain fragment variable (scFv) molecule that recognizes human CD19 (SEQ ID NO: 25), one is parts of the human CD28 protein (SEQ ID NO: 23) and the remaining part is the intracellular domain of human CD3ζ (SEQ ID NO: 21). The three functional domains are arranged in tandem, leading by a signaling peptide derived from human granulocyte-macrophage colony-stimulating factor (GM-CSF) receptor α-chain (i.e., colony stimulating factor 2 receptor subunit alpha (CSF2RA) signal peptide, the animo acid sequence set forth as SEQ ID NO: 19). The exact sequence of CD28 starts from 1114 and the MYPPPY motif, parts of its extracellular domain which can bind to CD80 and CD86 is included in the sequence. The transmembrane domain and the whole cytoplasmic tail of CD28 are also included. Nucleotides encoding CAR protein were codon optimized with internal algorithms and translated through a coxsackievirus B3 (CVB3) internal ribosome entry sites (IRESs) (SEQ ID NO: 17). Murine CD19 targeting CAR consists of the light chain variable region of the 1D3 antibody, a linker peptide (GGGGS)₃, the heavy chain variable region of the 1D3 antibody, a portion of the murine CD28 molecule and the cytoplasmic region of the murine CD3ζ molecule (SEQ ID NO: 29) (FIG. 1 bottom). A BspQ I or Pme I restriction endonuclease digestion site was inserted 3′ downstream of the element required for plasmid linearization.

### Example 2 Production of circRNA-CAR by Group I Intron Self-Splicing Reaction

The codon optimized nucleotide sequences were synthesized by Azenta Life Sciences. The Anabaena Group I intron permuted-intron-exon DNA construct sequence were provided in SEQ ID NO: 27 or 28, which consisted of complementary homology arms at both ends facilitating the folding of active ribozyme structure. The synthesized fragments were cloned into Spe I/Age I site of the pUC57 plasmid and linearized by Pme I (NEB, R0560L) at 37 °C for 2 hours (h). The linearized plasmid was purified by a DNA Clean & Concentrator™-25 kit (ZYMO RESEARCH, D4034) and proceeded to precursor RNA synthesis. The precursor RNA was synthesized by in vitro transcription using T7 RNA polymerase (Vvazyme, DD4101). In an IVT reaction system, reaction components were assembled with 400 ng linearized DNA template, 100 U T7 RNA Polymerase (Vvazyme, DD4101), 100 μM NTP solution mix, 10× reaction buffer (Vazyme. DD4101R), 10 U Rnase Inhibitor (Vazyme, DD4102-PA-01) and 0.008U pyrophosphatase (Vazyme, DD4103-PC-01), then program the thermocycler for the IVT reaction by incubation at 37 °C for 2 hours. After in vitro transcription, the IVT reaction was treated with 10 U Dnase I (Vazyme, EN401), and incubation at 37 °C for 30 mins to remove the linearized DNA template. After Dnase I treatment, the reactions were heated at 55 °C for 15 mins to get as many transesterification reactions as possible, RNA was then column purified by Monarch® RNA Cleanup Kit (NEB, T2040L).

### Example 3 CircRNA-CAR Prepared by T4 Rnl2

Briefly, the plasmid was linearized by digestion with BspQ I restriction endonuclease (Vazyme, DD4302-PC-02), the reaction was incubated at 50 °C for 3 hours and the linear DNA template was purified using DNA Clean & Concentrator-25 Kit (ZYMO RESEARCH, D4034). Following the linearization step, an aliquot of the linearized DNA template was added to an IVT reaction to get the precursor RNA. In an IVT reaction system, reaction components were assembled with 400 ng linearized DNA template, 100 U T7 RNA Polymerase (Vazyme, DD4101-PC-03), a NTP solution mix including 100 μM GTP (Vazyme, DD4108-PA-02), 600 μM GMP solution, 100 μM ATP (Vazyme, DD4106-PA-02), 100 μM CTP (Vazyme, DD4107-PA-02), 100 μM UTP (Vazyme, DD4105-PA-02), 10×reaction buffer (Vazyme, DD4101R), 10 U Rnase Inhibitor (Vazyme, DD4102-PA-01) and 0.008U Pyrophosphatase (Vazyme, DD4103-PC-01), and the IVT reaction was incubated at 37 °C for 2 hours. Then the linear products were exposed to Dnase I (Vazyme, EN401) digestion to remove the template DNA, and the RNA transcripts were column purified by Monarch® RNA Cleanup Kit (NEB, T2040L). To get the circular RNA, we then used T4 Rnl2 (Kactus, TRL-BE103-C1) to circularize the RNA transcripts, an enzyme ligation reaction was assembled, 10 U T4 RNA Ligase II, 10×reaction buffer and 10 μg linear RNA were added to a 40 μL reaction mixture and the reaction vessels was incubated at 25 °C for 2 hours. At last Rnase R exonuclease (Novoprotein, GMP-E224-M001) were added to degrade the linear RNA and enrich the circular product, 1 μg column purified RNA product, 1 U Rnase R was assembled to the 6 μl reaction system, and the reactions were incubated at 37 °C for 30 minutes. The circular RNA products were then purified using Monarch® RNA Cleanup Kit (NEB, T2040L).

### Example 4 CircRNA-LNP/fLNP Preparation

Rnase R enriched circRNAs were formulated into lipid nanoparticles (LNPs) with ionizable lipid SM102 (Cat: 06040008800, sinopeg). Briefly, an ethanolic lipid mixture of ionizable cationic lipid SM-102, DSPC, cholesterol, and DMG-PEG2000 was mixed with an aqueous solution containing the mRNA at acidic pH by NanoAssemblr™ Ignite™ nanoparticle formulation systems.

To prepare fabricated LNP (fLNP), circRNAs were encapsulated into LNP at first. And then LNP were conjugated with purified anti-human CD5, clone 5D7 antibodies. LNPs were prepared via microfluidic mixing with NanoAssemblr™ Ignite™ nanoparticle formulation systems. Briefly, an ethanolic lipid mixture was prepared consisting ionizable cationic lipid, phosphatidylcholine, cholesterol, polyethylene glycol-lipid and functionalized maleimide PEG-lipid at a ratio of 50:10:38:1.5:0.5 and then rapidly mixed with an aqueous solution containing the circRNA at acidic pH. The aqueous to ethanol volume ratio was 3:1. LNPs containing functionalized maleimide PEG-lipid were dialyzed at 4 °C overnight in PBS with 10 mM EDTA. The purified anti-human CD5, clone 5D7 antibodies were first reduced with 5 eq. of TCEP (Sigma-Aldrich) in 5 mM PBS at 37 °C for 1 hour with gentle shaking allowing conjugation to maleimide. Excess TCEP was removed by passing antibody solution through a Zeba 7 MWCO desalting column (ThermoFisher). Concentration of reduced antibody was quantified via absorbance at 280 nm via NanoDrop. The reactive sulfhydryl group on the antibody was then conjugated to functionalized LNPs for one hour at room temperature with gentle shaking. fLNPs were purified and concentrated using Amicon® Ultra-15 Centrifugal Filter Unit (MilliporeSigma). circRNA content was calculated by performing RiboGreen RNA assay (Thermo Fisher). Finally, fLNPs were stored at 4 °C prior to injection.

### Example 5 In Vitro Expression of Anti-CD19 CAR Encoding circRNA

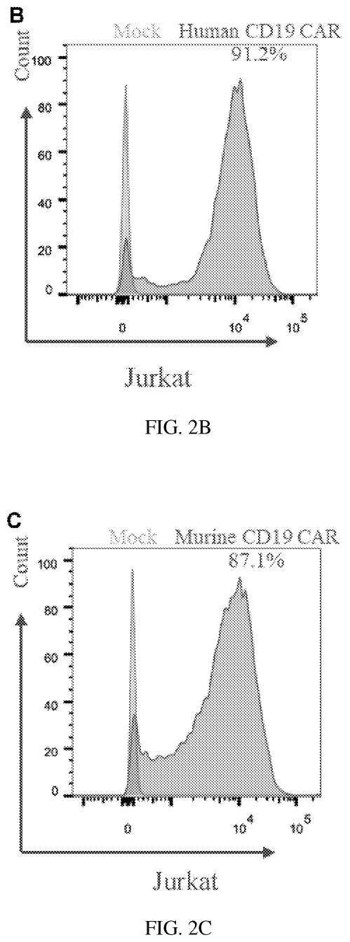

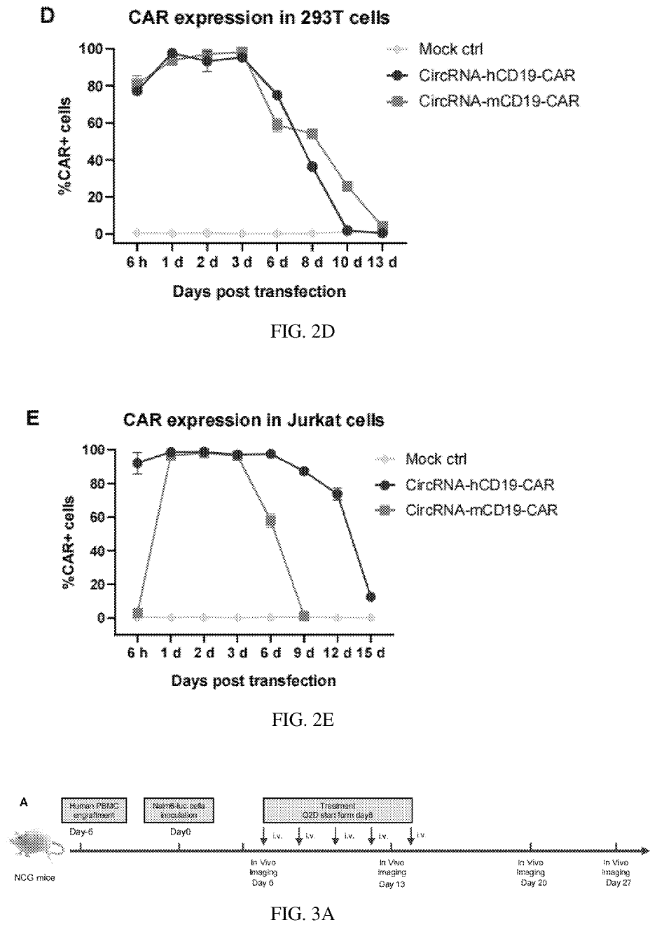

HEK293T cells were transfected with formulated circRNA-LNP (SEQ ID NO: 9 for anti-hCD19 CAR and SEQ ID NO: 29 for anti-mCD19 CAR). Fluorescein 5-isothiocyanate (FITC) labeled Monoclonal Anti-FMC63 Antibody (Acrobiosystems, FM3-FY45G0) and PE-Labeled Recombinant Protein L (Acrobiosystems, RPL-PP2H2) were used for cell surface staining 12 h post-transfection for transient expression analysis. It is shown that about 90% cells expressed CARs on the cell surface (FIG. 2A-2C). CircRNA-LNPs were transfected into Jurkat or HEK293T cells with Fetal Bovine Serum (FBS) reduced medium (2.5%) to evaluate the persistence of anti-CD19 CAR. The expression of anti-hCD19 CAR was sustained for ~2 weeks on Jurkat cells and ~1 week on HEK293T cells. Expression of anti-murine CD19 CAR sustained ~1 week in both Jurkat and HEK293T cells and gradually declined until 9 days after transfection (FIG. 2D-2E).

### Example 6 Anti-hCD19 CAR circRNA Treatment Showed Sustained Anti-Tumor Efficacy In Vivo

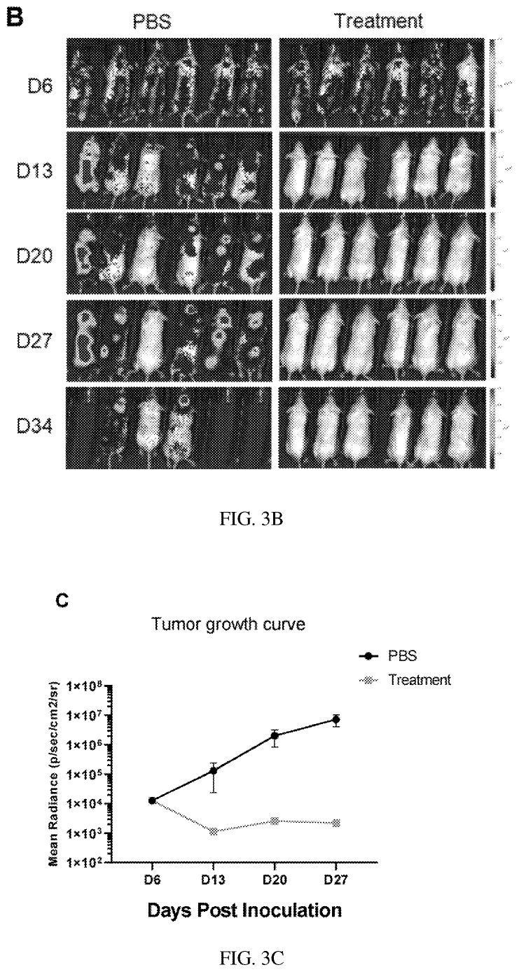

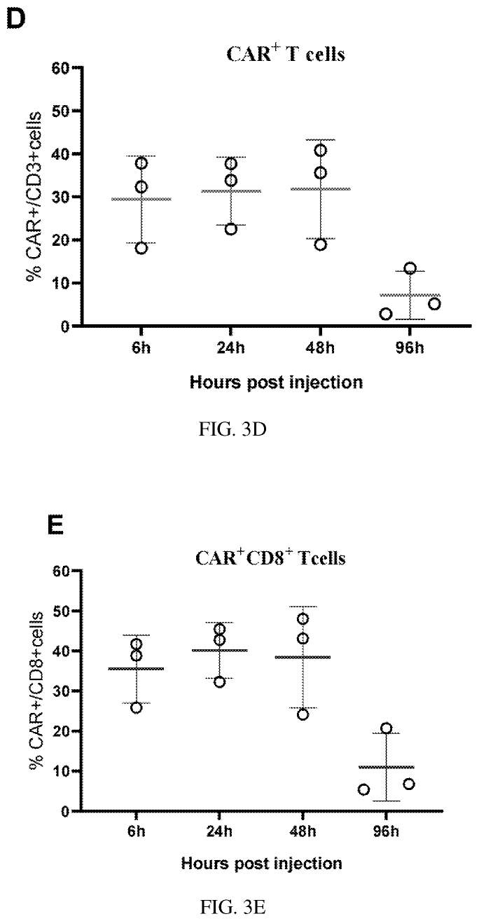

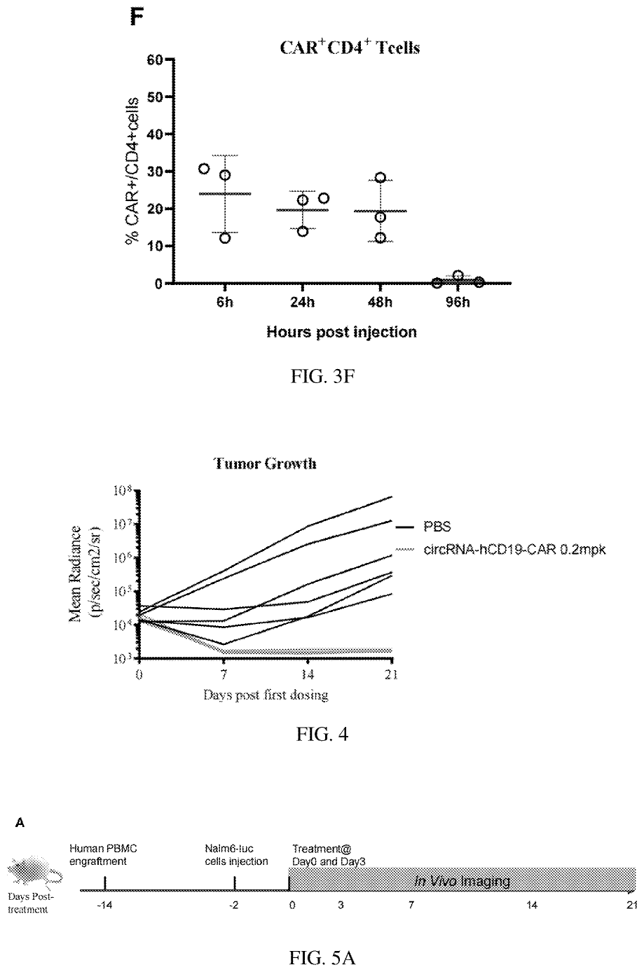

To test the function of anti-hCD19 CAR circRNA (SEQ ID NO: 9) in vivo, we develop a humanized mouse model bearing Nalm6-luciferase expressing tumor cells to monitor human B cell leukemia. hPBMCs-NCG-Nalm6-luc mice were generated by intravenous injection of 1×10⁷ human peripheral blood mononuclear cells (PBMCs) into 6 weeks old female NCG mice (Gempharmatech, Nanjing, China). The sever immune deficient niche allows for efficient transplantation of human PBMCs. Six days later, 5×10⁵ human B cell leukemia cell line Nalm6 cells which constitutively expressing luciferase were inoculated by intravenous injection (FIG. 3A). This technology features luciferase enzymes and luciferin so that bioluminescent flux can be detected in tissues and tumor cells growth can be visualized in live organisms. The engraftment efficiency of human immune cells (hCD45⁺) was determined 10 days post PBMCs injection by flow cytometry analysis of blood cells. A success mouse model was designated as more than 1×10⁴ human T cells reconstituted in the peripheral blood and moderate luciferase photon signals ranging from 5×10³ to 1.5×10⁴ p/sec/cm2/sr.

hPBMCs-NCG-Nalm6-luc recipient mice were randomized into control and treatment groups. CircRNA-LNP (SEQ ID NO: 9) were administrated by intravenous injection of 20 μg/dose every other day (Q2D). All mice were treated 5 doses in total. Tumor growth was monitored by the IVIS® Spectrum in vivo imaging system every week (FIG. 3A). Mice treated with anti-hCD19 CAR circRNA (SEQ ID NO: 9) showed durable tumor control with almost completely tumor cells eradication and lasted for a long time after the withdrawal of the treatment, while tumors of the control group showed sustained progression (FIG. 3B-3C).

To investigate the efficiency of in vivo generation of CAR-T cells, peripheral blood samples were analyzed for CAR expression in hPBMC-M-NSG humanized mice. Mice were administrated with 20 μg anti-hCD19 CAR circRNA, and peripheral blood was proceeded to flow cytometry analysis with FITC labeled anti-FMC63 antibodies (Acrobiosystems, FM3-FY45G0) at indicated timepoint. It is shown that ~30% of T cells were stained as CAR positive cells. A single dose of anti-hCD19 CAR circRNA remained effective 48 h post injection and sustained to ~4 days, demonstrating successful in vivo generation of CAR-T cells (FIG. 3D-3F).

### Example 7 Anti-hCD19 CAR circRNA is Efficacious at Low Doses and Frequency

Based on the robust anti-tumor efficacy of anti-hCD19 CAR circRNA hPBMCs-NCG-Nalm6-luc recipient mice were treated with lower dose and less frequency. Total tumor control was still observed with doses as low as 0.2 mg/kg for 2 doses (FIG. 4), which has never been achieved by mRNA CAR.

### Example 8 Efficacy of Anti-B Cell Leukemia with fLNP Formulated circRNA

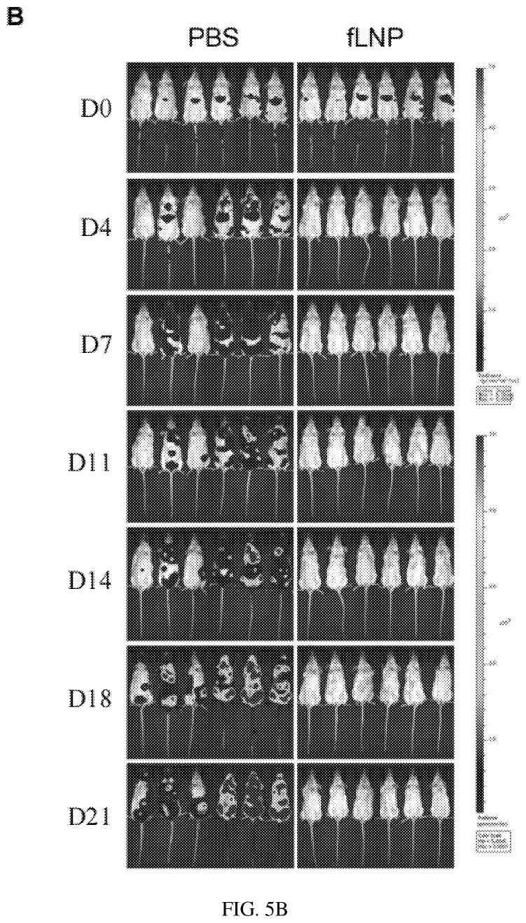

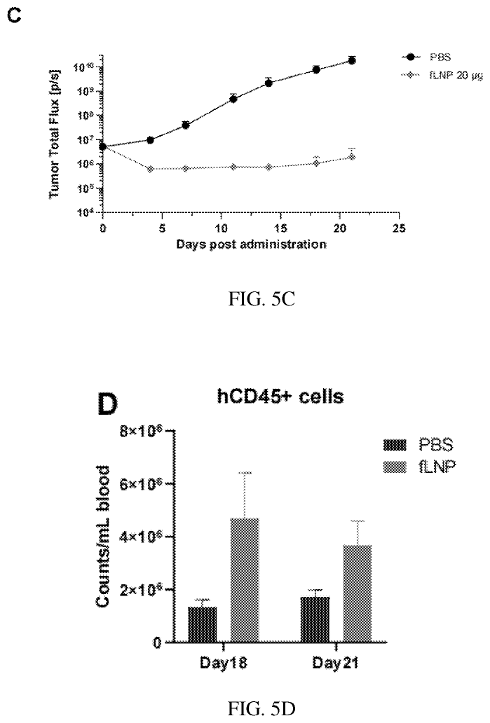

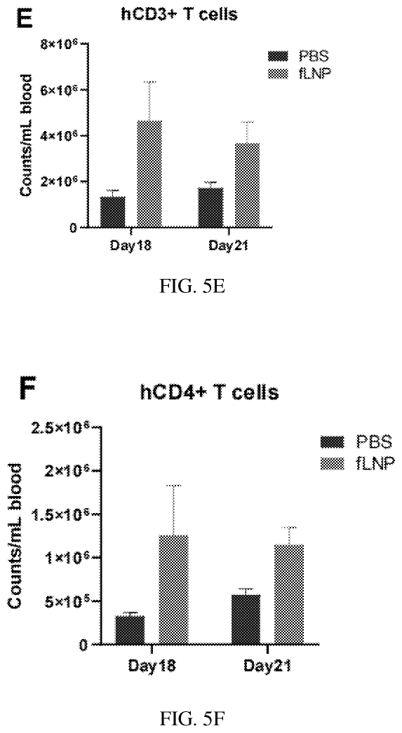

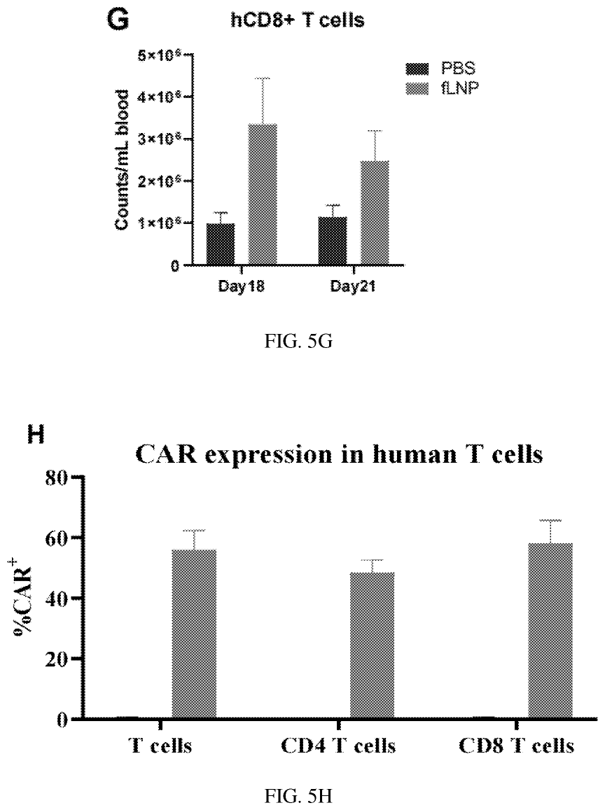

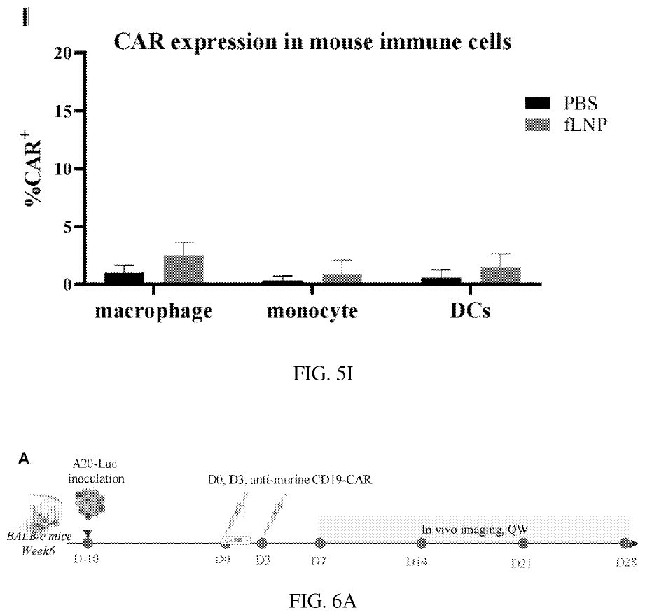

Anti-tumor efficacy of anti-hCD19 CAR circRNA was studied in hPBMCs-M-NSG-Nalm6-Luc tumor model. 6- to 8-week-old M-NSG mice (Shanghai Model Organisms, Shanghai, China) were inoculated with 1×10⁷ human PBMCs, Nalm6 cells expressing luciferase were injected intravenously 12 days after hPBMCs injection. Two days later, mice were randomized and treated with anti-hCD19 CAR circRNA via CD5 targeted fLNP. Mice were administrated with 1 mg/kg for two doses at Day0 and Day3, tumor burden were monitored twice a week by IVIS (FIG. 5A). Mice treated with anti-hCD19 CAR circRNA exhibited significantly lower tumor luminescence in fLNP group compared with that of the control group (FIG. 5B-5C). Immune cells population and CAR expression in the peripheral blood were analyzed with flow cytometry. 18-days post treatment initiation, immune cell counts, CD3+ T cells, CD4+ T cells and CD8+ T cells were all increased in treatment groups compared to control group (FIG. 5D-5G), which indicate T cells expansion after circRNA treatment. High level of CAR expression in human T cells can be detected in fLNP treatment group, while rare expression of CARs on mouse immune cells were detected (FIG. 5H-5I).

### Example 9 Anti-Murine CD19 CAR circRNA Treatment Delayed Tumor Progression in A20 Syngeneic Model

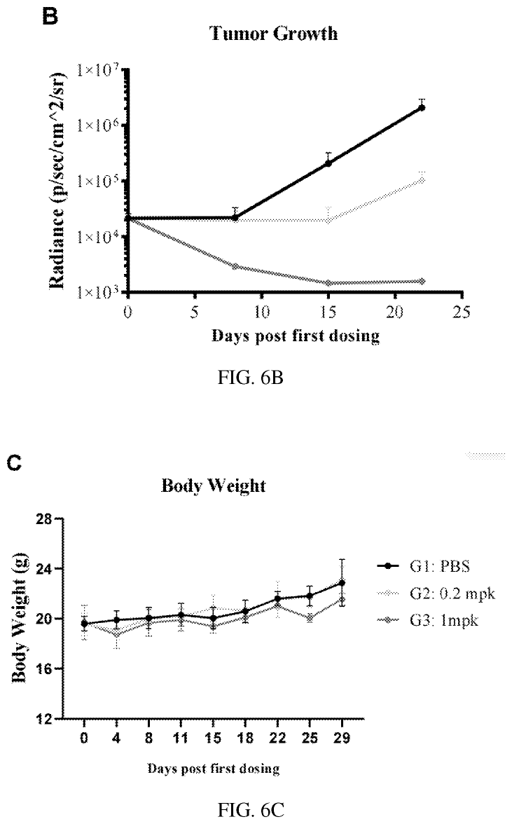

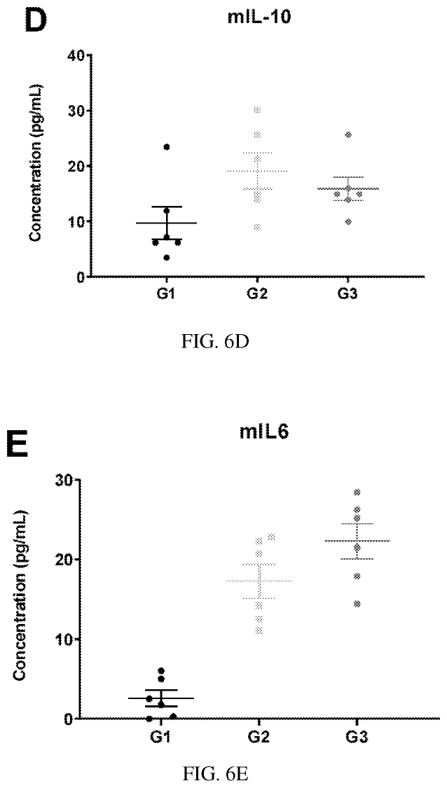

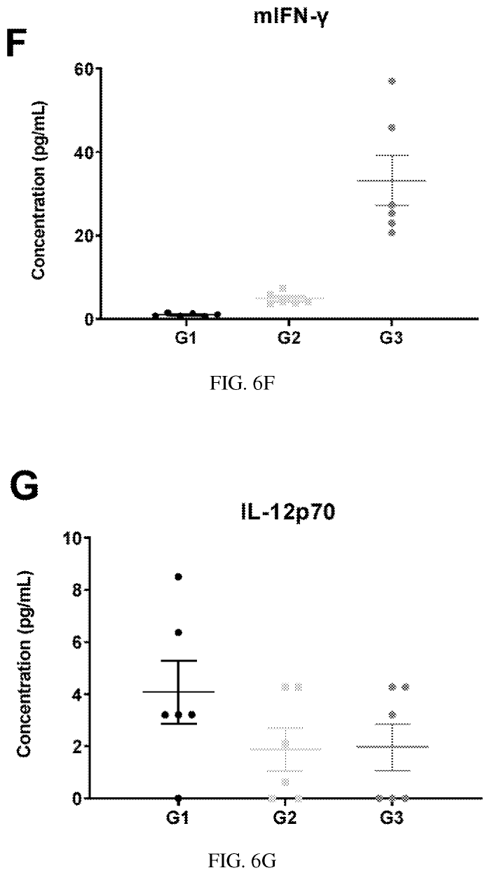

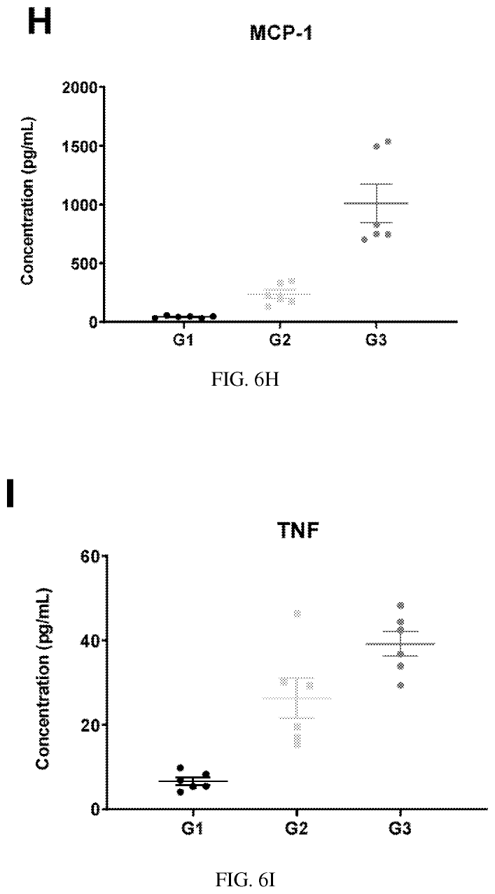

Anti-tumor efficacy of anti-mCD19 CAR circRNA was evaluated in an orthotopic leukemia mouse model based upon the A20 cell line. Female BALB/c mice were transplanted with 2×10⁶ A20-luc leukemia cells via intravenous injection. 9 days post A20-luc cells inoculation, mice were randomized and administrated with anti-mCD19 CAR circRNA at Day0, Day3 and Day6. Tumor burden was measured by in vivo imaging every week (FIG. 6A). The results showed that anti-mCD19 CAR circRNA treatment inhibits A20 tumor cell growth in a dose-dependent manner (FIG. 6B) with no significant difference in body weight change (FIG. 6C).

Cytokine release syndrome is the most concerned safety issue of CAR-T cell therapies. Based on dose-dependent anti-tumor effect on BALB/c-A20 syngeneic model, cytokine release was evaluated at this immunocompetent mouse model. Serum cytokines were determined at Day4 (12 h post last administration). A BD™ Cytometric Bead Array (CBA) Mouse Inflammation Kit (BD Biosciences, 552364) was used to determine the cytokines in the serum of anti-murine CD19 CAR circRNA treated mice. IFN-γ, IL-10, TNF, MCP-1, IL-6 were all elevated in a dose-dependent manner but to a manageable extent after injection of anti-hCD19 CAR circRNA (FIG. 6D-6I).

### Example 10 Applying of Anti-mCD19 CAR circRNA Therapy to SLE Treatment in Mouse Disease Model

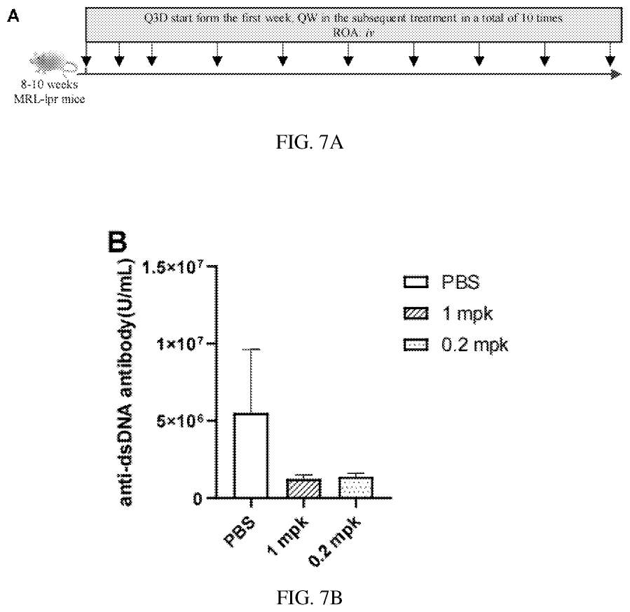

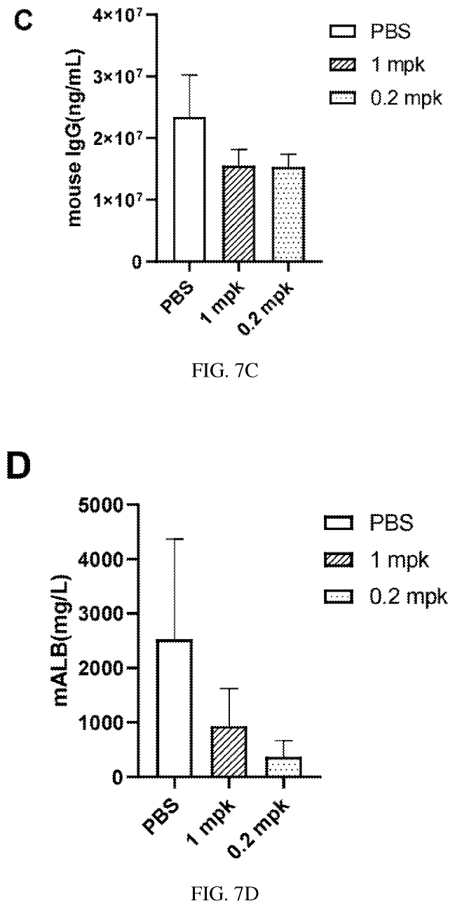

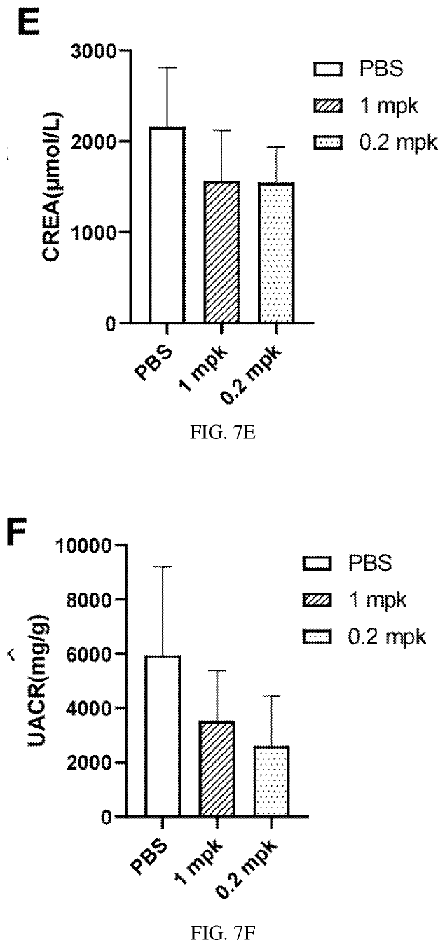

LNP encapsulated anti-mCD19 CAR-circRNA were transferred to in MRLfas/fas (MRL-lpr) mice before the onset of disease to determine their role in SLE prevention. 8-10 weeks old female MRL-lpr mice were administrated with 1 mg/kg or 0.2 mg/kg anti-mCD19 CAR circRNA via intravenous injection every three days in the first week, and three times later, all mice received circRNAs treatment once a week (FIG. 7A). To evaluate the therapeutic function of circRNA-CAR, we determined serum anti-dsDNA autoantibody titers and total IgG using enzyme-linked immunosorbent assay (ELISA) in circRNA-treated and control mice at Day63 and Day77 respectively (FIGS. 7B-7C). Proteinuria and urine creatinine were also detected to monitor kidney injury at Day 70 (FIGS. 7D-7E). These results show that circRNA encoded anti-CD19 CAR was effective in the prevention and treatment of a murine model of SLE, indicating its potential for clinical use in patients.

### Example 11 CircRNA-CAR Induced Potent B-Cell Depletion in Non-Human Primates (NHPs)

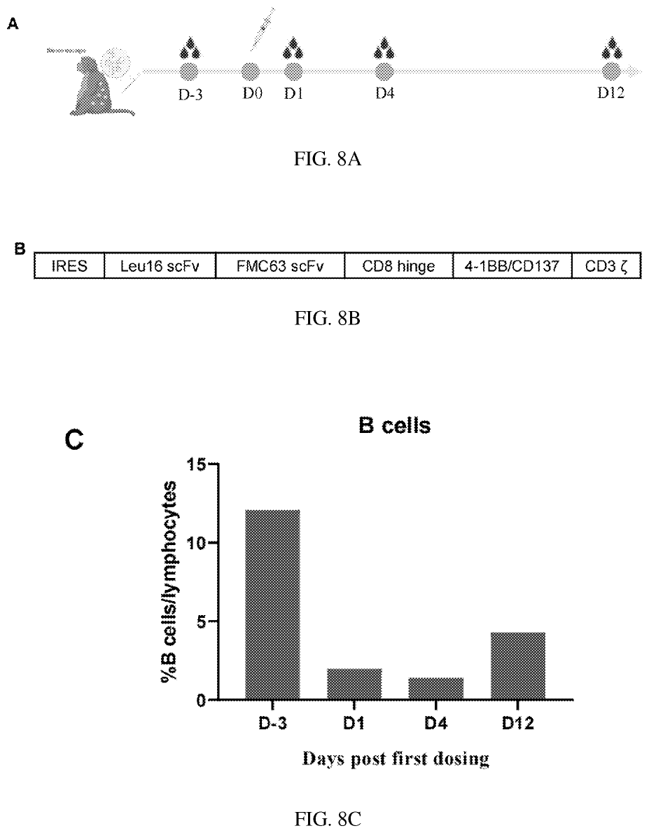

The effect of in vivo CAR on B cell depletion was further assessed in non-human primates. Male rhesus macaque (Macaca mulatta) was administrated intravenously with anti-CD20/CD19 bispecific CARs (SEQ ID NO: 31 for amino acid sequence and SEQ ID NO: 32 for nucleotide sequence) at Day0 and Day14. The construct was generated by linking the single chain fragment variable (scFv) region of Leu-16 (CD20) and monoclonal antibody FMC-63 (CD19) with a CD8 hinge and transmembrane domain to the intracellular 4-1BB (CD137) and CD35 signaling domains (FIG. 8B). The percentage of lymphocytes was determined pre- and post-intravenous circRNA injection by flow cytometry. It is shown that B cells in the periphery blood were decreased by 84% at Day 1 following circRNA injection, and maintained at a low level at least to Day4. Then, the B cell level raised slowly, and reached to 33% of pre-treatment B cell levels at Day 12 (FIG. 8C). The results indicate a very quick and sustainable B cell depletion in NHPs.

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof.

All patents, patent applications, and publications cited herein are fully incorporated by reference herein in their entirety.

1. A circular RNA encoding chimeric antigen receptor (CAR),
wherein the circular RNA comprises a regulatory element and an expression element comprising a polynucleotide encoding chimeric antigen receptor (CAR) or a variant thereof; and
the chimeric antigen receptor (CAR) comprises an antigen binding domain, a transmembrane domain, a co-stimulatory domain, and an intracellular T cell signaling domain;
wherein each element is optionally connected by a polynucleotide encoding a linker.

2. The circular RNA of claim 1, wherein the polynucleotide encoding CAR or a variant thereof has at least 50%, at least 60%, at least 70%, at least 80%, at least 90%, at least 95%, at least 98%, at least 99% or 100% identity with the sequence set forth as SEQ ID NO: 9, 10, 11, 12, 13, 14, 15, 16 or 30.

3. The circular RNA of claim 2, wherein an amino acid sequence or a variant thereof of the CAR encoded by the polynucleotide has at least 50%, at least 60%, at least 70%, at least 80%, at least 90%, at least 95%, at least 98%, at least 99% or 100% identity with the sequence set forth as SEQ ID NO: 1, 2, 3, 4, 5, 6, 7, 8 or 29.

4. The circular RNA of claim 1, wherein the antigen binding domain is one or more antigen binding domain(s) specific for CD19, CD20, BCMA, CLDN6, CLDN18.2, ERBB2 (HER2), PSMA, SLAMF3, SLAMF7, CD66c, MSLN, CD38, CD123, GPC3, EGFRvIII, CD171, MUC1 and/or GPRC5D.

5. The circular RNA of claim 4, wherein the antigen binding domain is a single chain variable fragment (scFv) specific for CD19, CD20 and/or BCMA.

6. The circular RNA of claim 5, wherein the amino acid sequence of the scFv has at least 50%, at least 60%, at least 70%, at least 80%, at least 90%, at least 95%, at least 98%, at least 99% or 100% identity with the sequence set forth as SEQ ID NO: 25, 33, 35, 37, 39 or 41; or
wherein the amino acid sequence of the scFv is encoded by a polynucleotide sequence having at least 50%, at least 60%, at least 70%, at least 80%, at least 90%, at least 95%, at least 98%, at least 99% or 100% identity with the sequence set forth as SEQ ID NO: 26, 34, 36, 38, 40 or 42.

7. The circular RNA of claim 1, wherein the co-stimulatory domain is selected from the group consisting of CD28, 4-1BB, OX40, CD27, CD30, ICOS, GITR, CD40, CD2, SLAM, and combinations thereof.

8. The circular RNA of claim 1, wherein the transmembrane domain is selected from the group consisting of CD28 and CD8 transmembrane domain.

9. The circular RNA of claim 1, wherein the intracellular T cell signaling domain is CD3zeta signaling domain.

10. The circular RNA of claim 1, wherein the regulatory element comprises a polynucleotide encoding an internal ribosomal entry site (IRES) or a fragment thereof.

11. The circular RNA of claim 10, wherein the IRES is selected from the group consisting of Coxsackievirus B3 (CVB3) IRES, Enterovirus 71 (EV71) IRES, encephalomyocarditis virus (EMCV) IRES, picornavirus (PV) IRES, hepatitis C virus (HCV) IRES, adenovirus (AdV) IRES, human papillomavirus type 31 (HPV31) IRES, human herpesvirus (HHV) IRES, Rous sarcoma virus (RSV) IRES, classical swine fever virus (CSFV) IRES, FGF9 IRES, SLC7A1 IRES, and RUNX1 IRES.

12. The circular RNA of claim 1, comprising a regulatory element and an expression element comprising a polynucleotide encoding a bispecific CAR specific for CD20 and CD19,
wherein the scFv of the bispecific CAR specific for CD20 has an amino acid sequence having at least 50%, at least 60%, at least 70%, at least 80%, at least 90%, at least 95%, at least 98%, at least 99% or 100% identity with the sequence set forth as the amino acid sequence selected from SEQ ID NO: 25, 33, or 35; and/or, wherein the scFv of the bispecific CAR specific for CD19 has an amino acid sequence having at least 50%, at least 60%, at least 70%, at least 80%, at least 90%, at least 95%, at least 98%, at least 99% or 100% identity with the sequence set forth as SEQ ID NO:41;
wherein each element is optionally connected by a polynucleotide encoding a linker.

13. The circular RNA of claim 12, wherein the polynucleotide encoding CAR or a variant thereof has at least 50%, at least 60%, at least 70%, at least 80%, at least 90%, at least 95%, at least 98%, at least 99% or 100% identity with the sequence set forth as SEQ ID NO: 32; or
wherein an amino acid sequence or a variant thereof of the CAR encoded by the polynucleotide has at least 50%, at least 60%, at least 70%, at least 80%, at least 90%, at least 95%, at least 98%, at least 99% or 100% identity with the sequence set forth as SEQ ID NO: 31.

14. A composition comprising the circular RNA of the claim 1, wherein the composition comprises pharmaceutically acceptable excipients.

15. The composition of claim 14, further comprising nanoparticle.

16. The composition of claim 15, wherein lipid nanoparticle comprises at least one of an ionizable lipid, a structural lipid, and a PEG-modified lipid.

17. A method for immunotherapy in a subject, comprising administering a therapeutically effective amount of the circular RNA of claim 1 to the subject.

18. The method of claim 17, wherein the immunotherapy is selected from treating cancer, hyperproliferative disease, fibrosis or autoimmune diseases.

19. A linear RNA polynucleotide comprising a regulatory element comprises a polynucleotide encoding an internal ribosomal entry site (IRES) or a fragment thereof, an expression element encoding a chimeric antigen receptor (CAR) polypeptide, and at least one self-circularizing element;
wherein each element is optionally connected by a polynucleotide encoding a linker,
wherein the chimeric antigen receptor (CAR) comprises an antigen binding domain, a transmembrane domain, a co-stimulatory domain, and an intracellular T cell signaling domain, and
wherein the IRES is selected from the group consisting of Coxsackievirus B3 (CVB3) IRES, Enterovirus 71 (EV71) IRES, encephalomyocarditis virus (EMCV) IRES, picornavirus (PV) IRES, hepatitis C virus (HCV) IRES, adenovirus (AdV) IRES, human papillomavirus type 31 (HPV31) IRES, human herpesvirus (HHV) IRES, Rous sarcoma virus (RSV) IRES, classical swine fever virus (CSFV) IRES, FGF9 IRES, SLC7A1 IRES, and RUNX1 IRES.

20. The linear RNA polynucleotide of claim 19, wherein the polynucleotide encoding CAR or a variant thereof has at least 50%, at least 60%, at least 70%, at least 80%, at least 90%, at least 95%, at least 98%, at least 99% or 100% identity with the sequence set forth as SEQ ID NO: 9, 10, 11, 12, 13, 14, 15, 16 or 30.

21. The linear RNA polynucleotide of claim 20, wherein an amino acid sequence or a variant thereof of the CAR encoded by the polynucleotide has at least 50%, at least 60%, at least 70%, at least 80%, at least 90%, at least 95%, at least 98%, at least 99% or 100% identity with the sequence set forth as SEQ ID NO: 1, 2, 3, 4, 5, 6, 7, 8 or 29.

22. The linear RNA polynucleotide of claim 19, wherein the antigen binding domain is one or more antigen binding domain(s) specific for CD19, CD20, BCMA, CLDN6, CLDN18.2, ERBB2 (HER2), PSMA, SLAMF3, SLAMF7, CD66c, MSLN, CD38, CD123, GPC3, EGFRvIII, CD171, MUC1 and/or GPRC5D.

23. The linear RNA polynucleotide of claim 22, wherein the antigen binding domain is a single chain variable fragment (scFv) specific for CD19, CD20 and/or BCMA.

24. The circular RNA of claim 23, wherein the amino acid sequence of the scFv has at least 50%, at least 60%, at least 70%, at least 80%, at least 90%, at least 95%, at least 98%, at least 99% or 100% identity with the sequence set forth as SEQ ID NO: 25, 33, 35, 37, 39 or 41; or
wherein the amino acid sequence of the scFv is encoded by a polynucleotide sequence having at least 50%, at least 60%, at least 70%, at least 80%, at least 90%, at least 95%, at least 98%, at least 99% or 100% identity with the sequence set forth as SEQ ID NO: 26, 34, 36, 38, 40 or 42.

25. The linear RNA polynucleotide of claim 19, wherein the co-stimulatory domain is selected from the group consisting of CD28, 4-1BB, OX40, CD27, CD30, ICOS, GITR, CD40, CD2, SLAM, and combinations thereof,
wherein the transmembrane domain is selected from the group consisting of CD28 and CD8 transmembrane domain, and/or,
wherein the intracellular T cell signaling domain is CD3zeta signaling domain.

26. The linear RNA polynucleotide of claim 19, comprising, in the following order from 5′ to 3′:
optionally a 5′-self-circularizing element;
the regulatory element comprises a polynucleotide encoding an internal ribosomal entry site (IRES) or a fragment thereof;
optionally a signal peptide element;
the expression element comprising a polynucleotide encoding chimeric antigen receptor (CAR);
optionally a stop codon;
optionally a 3′-self-circularizing element.

27. The linear RNA polynucleotide of claim 26, wherein
the 5′-self-circularizing elements comprises a 5′-Group I intron fragment and an Exon2; and
the 3′-self-circularizing elements comprises an Exon1 and 3′-Group I intron fragment.

28. The linear RNA polynucleotide of claim 26, wherein the self-circularizing element is for T4 RNA ligase 2 circulation, and wherein the 5′ end of the linear RNA polynucleotide is phosphorylated and the 3′ end of the linear RNA polynucleotide is OH.

29. A DNA vector suitable for synthesizing the linear RNA polynucleotide of claim 19.

30. A population of eukaryotic cells comprising a circular RNA polynucleotide of claim 1, wherein the population of eukaryotic cells express the CAR complex protein encoded by the circular RNA polynucleotide on its cell surface.
